SEQUENCE LISTING (124 sequences)

SEQ ID NO: 1 - DNA, 657 bp, Physcomitrella patens
  CDS (1)..(657): NF-YB transcription factor (EST 265)
atggccgatagctacggtcacaacgcaggttcaccagagagcagcccgcattctgataacgagtccgggggtcattaccgagaccaggatgcttctgtacgggaacaggatcggttcctgcccatcgcgaacgtgagccgaatcatgaagaaggcgttgccgtctaatgcaaaaatttcgaaggacgcgaaagagactgtgcaggagtgtgtgtccgagttcatcagcttcatcactggtgaggcgtcagataagtgccagagggagaagagaaagacgatcaacggtgacgacttgctgtgggccatgagtacacttggtttcgaagattacgtggagcctctgaaggtttacctacacaaataccgggagctagagggagagaaggcttccacggccaagggtggtgaccagcaaggagggaaagaagggagtcaaggtgttatggggtccatgggtatgtcgggcggaatgaacggtatgaacggtacgatgaacgggaatatgcatggacatggaatcccggtgtcgatgcagatgctgcagcagtcgtacggacagcaggcacctccagggatgatgtattcccctcatcagatgatgccgcaataccagatgccaatgcagtctggtggaaaccagcctcgtggagtgtag

SEQ ID NO: 2 - protein, 218 aa, Physcomitrella patens
MADSYGHNAGSPESSPHSDNESGGHYRDQDASVREQDRFLPIANVSRIMKKALPSNAKISKDAKETVQECVSEFISFITGEASDKCQREKRKTINGDDLLWAMSTLGFEDYVEPLKVYLHKYRELEGEKASTAKGGDQQGGKEGSQGVMGSMGMSGGMNGMNGTMNGNMHGHGIPVSMQMLQQSYGQQAPPGMMYSPHQMMPQYQMPMQSGGNQPRGV

SEQ ID NO: 3 - DNA, 573 bp, Arabidopsis thaliana
  CDS (1)..(573): NF-YB transcription factor (AT5G47640)
atgggggattccgacagggattccggtggagggcaaaacgggaacaaccagaacggacagtcctccttgtctccaagagagcaagacaggttcttgccgatcgctaacgtcagccggatcatgaagaaggccttgcccgccaacgccaagatctctaaagatgccaaagagacgatgcaggagtgtgtctccgagttcatcagcttcgtcaccggagaagcatctgataagtgtcagaaggagaagaggaagacgatcaacggagacgatttgctctgggctatgactactctaggttttgaggattatgttgagccattgaaagtttacttgcagaggtttagggagatcgaaggggagaggactggactagggaggccacagactggtggtgaggtcggagagcatcagagagatgctgtcggagatggcggtgggttctacggtggtggtggtgggatgcagtatcaccaacatcatcagtttcttcaccagcagaaccatatgtatggagccacaggtggcggtagcgacagtggaggtggagctgcctccggtaggacaaggacttaa

SEQ ID NO: 4 - protein, 190 aa, Arabidopsis thaliana
MGDSDRDSGGGQNGNNQNGQSSLSPREQDRFLPIANVSRIMKKALPANAKISKDAKETMQECVSEFISFVTGEASDKCQKEKRKTINGDDLLWAMTTLGFEDYVEPLKVYLQRFREIEGERTGLGRPQTGGEVGEHQRDAVGDGGGFYGGGGGMQYHQHHQFLHQQNHMYGATGGGSDSGGGAASGRTRT

SEQ ID NO: 5 - DNA, 558 bp, Zea mays
  CDS (1)..(558): NF-YB transcription factor (NM_001112582.1)
atggcggaagctccggcgagccctggcggcggcggcgggagccacgagagcgggagccccaggggaggcggaggcggtggcagcgtcagggagcaggacaggttcctgcccatcgccaacatcagtcgcatcatgaagaaggccatcccggctaacgggaagaccatcccggctaacgggaagatcgccaaggacgctaaggagaccgtgcaggagtgcgtctccgagttcatctccttcatcactagcgaagcgagtgacaagtgccagagggagaagcggaagaccatcaatggcgacgatctgctgtgggccatggccacgctggggtttgaagactacattgaacccctcaaggtgtacctgcagaagtacagagagatggagggtgatagcaagttaactgcaaaatctagcgatggctcaattaaaaaggatgcccttggtcatgtgggagcaagtagctcagctgcacaagggatgggccaacagggagcatacaaccaaggaatgggttatatgcaaccccagtaccataacggggatatctcaaactaa

SEQ ID NO: 6 - protein, 185 aa, Zea mays
MAEAPASPGGGGGSHESGSPRGGGGGGSVREQDRFLPIANISRIMKKAIPANGKTIPANGKIAKDAKETVQECVSEFISFITSEASDKCQREKRKTINGDDLLWAMATLGFEDYIEPLKVYLQKYREMEGDSKLTAKSSDGSIKKDALGHVGASSSAAQGMGQQGAYNQGMGYMQPQYHNGDISN

SEQ ID NO: 7 - DNA, 657 bp, Artificial
  synthetic chimeric sequence (NFYB-C1)
atggccgatagctacggtcacaacgcaggttcaccagagagcagcccgcattctgataacgagtccgggggtcattaccgagaccaggatgcttctccaagagagcaagacaggttcttgccgatcgctaacgtcagccggatcatgaagaaggccttgcccgccaacgccaagatctctaaagatgccaaagagacgatgcaggagtgtgtctccgagttcatcagcttcgtcaccggagaagcatctgataagtgtcagaaggagaagaggaagacgatcaacggagacgatttgctctgggctatgactactctaggttttgaggattatgttgagccattgaaagtttacttgcagaggtttagggagatcgaaggggagagggcttccacggccaagggtggtgaccagcaaggagggaaagaagggagtcaaggtgttatggggtccatgggtatgtcgggcggaatgaacggtatgaacggtacgatgaacgggaatatgcatggacatggaatcccggtgtcgatgcagatgctgcagcagtcgtacggacagcaggcacctccagggatgatgtattcccctcatcagatgatgccgcaataccagatgccaatgcagtctggtggaaaccagcctcgtggagtgtag

SEQ ID NO: 8 - protein, 218 aa, Artificial
  Synthetic Construct
MADSYGHNAGSPESSPHSDNESGGHYRDQDASPREQDRFLPIANVSRIMKKALPANAKISKDAKETMQECVSEFISFVTGEASDKCQKEKRKTINGDDLLWAMTTLGFEDYVEPLKVYLQRFREIEGERASTAKGGDQQGGKEGSQGVMGSMGMSGGMNGMNGTMNGNMHGHGIPVSMQMLQQSYGQQAPPGMMYSPHQMMPQYQMPMQSGGNQPRGV

SEQ ID NO: 9 - DNA, 597 bp, Artificial
  synthetic chimeric sequence (NFYB-C2)
atggccgatagctacggtcacaacgcaggttcaccagagagcagcccgcattctgataacgagtccgggggtcattaccgagaccaggatgcttctgtacgggaacaggatcggttcctgcccatcgcgaacgtgagccgaatcatgaagaaggcgttgccgtctaatgcaaaaatttcgaaggacgcgaaagagactgtgcaggagtgtgtgtccgagttcatcagcttcatcactggtgaggcgtcagataagtgccagagggagaagagaaagacgatcaacggtgacgacttgctgtgggccatgagtacacttggtttcgaagattacgtggagcctctgaaggtttacctacacaaataccgggagctagagggagagaagactggactagggaggccacagactggtggtgaggtcggagagcatcagagagatgctgtcggagatggcggtgggttctacggtggtggtggtgggatgcagtatcaccaacatcatcagtttcttcaccagcagaaccatatgtatggagccacaggtggcggtagcgacagtggaggtggagctgcctccggtaggacaaggacttaa

SEQ ID NO: 10 - protein, 198 aa, Artificial
  Synthetic Construct
MADSYGHNAGSPESSPHSDNESGGHYRDQDASVREQDRFLPIANVSRIMKKALPSNAKISKDAKETVQECVSEFISFITGEASDKCQREKRKTINGDDLLWAMSTLGFEDYVEPLKVYLHKYRELEGEKTGLGRPQTGGEVGEHQRDAVGDGGGFYGGGGGMQYHQHHQFLHQQNHMYGATGGGSDSGGGAASGRTRT

SEQ ID NO: 11 - DNA, 597 bp, Artificial
  synthetic chimeric sequence (NFYB-C3)
atggccgatagctacggtcacaacgcaggttcaccagagagcagcccgcattctgataacgagtccgggggtcattaccgagaccaggatgcttctccaagagagcaagacaggttcttgccgatcgctaacgtcagccggatcatgaagaaggccttgcccgccaacgccaagatctctaaagatgccaaagagacgatgcaggagtgtgtctccgagttcatcagcttcgtcaccggagaagcatctgataagtgtcagaaggagaagaggaagacgatcaacggagacgatttgctctgggctatgactactctaggttttgaggattatgttgagccattgaaagtttacttgcagaggtttagggagatcgaaggggagaggactggactagggaggccacagactggtggtgaggtcggagagcatcagagagatgctgtcggagatggcggtgggttctacggtggtggtggtgggatgcagtatcaccaacatcatcagtttcttcaccagcagaaccatatgtatggagccacaggtggcggtagcgacagtggaggtggagctgcctccggtaggacaaggacttaa

SEQ ID NO: 12 - protein, 198 aa, Artificial
  Synthetic Construct
MADSYGHNAGSPESSPHSDNESGGHYRDQDASPREQDRFLPIANVSRIMKKALPANAKISKDAKETMQECVSEFISFVTGEASDKCQKEKRKTINGDDLLWAMTTLGFEDYVEPLKVYLQRFREIEGERTGLGRPQTGGEVGEHQRDAVGDGGGFYGGGGGMQYHQHHQFLHQQNHMYGATGGGSDSGGGAASGRTRT

SEQ ID NO: 13 - DNA, 633 bp, Artificial
  synthetic chimeric sequence (NFYB-C4)
atgggggattccgacagggattccggtggagggcaaaacgggaacaaccagaacggacagtcctccttgtctccaagagagcaagacaggttcttgccgatcgctaacgtcagccggatcatgaagaaggccttgcccgccaacgccaagatctctaaagatgccaaagagacgatgcaggagtgtgtctccgagttcatcagcttcgtcaccggagaagcatctgataagtgtcagaaggagaagaggaagacgatcaacggagacgatttgctctgggctatgactactctaggttttgaggattatgttgagccattgaaagtttacttgcagaggtttagggagatcgaaggggagagggcttccacggccaagggtggtgaccagcaaggagggaaagaagggagtcaaggtgttatggggtccatgggtatgtcgggcggaatgaacggtatgaacggtacgatgaacgggaatatgcatggacatggaatcccggtgtcgatgcagatgctgcagcagtcgtacggacagcaggcacctccagggatgatgtattcccctcatcagatgatgccgcaataccagatgccaatgcagtctggtggaaaccagcctcgtggagtgtag

SEQ ID NO: 14 - protein, 210 aa, Artificial
  Synthetic Construct
MGDSDRDSGGGQNGNNQNGQSSLSPREQDRFLPIANVSRIMKKALPANAKISKDAKETMQECVSEFISFVTGEASDKCQKEKRKTINGDDLLWAMTTLGFEDYVEPLKVYLQRFREIEGERASTAKGGDQQGGKEGSQGVMGSMGMSGGMNGMNGTMNGNMHGHGIPVSMQMLQQSYGQQAPPGMMYSPHQMMPQYQMPMQSGGNQPRGV

SEQ ID NO: 15 - DNA, 573 bp, Artificial
  synthetic chimeric sequence (NFYB-C5)
atgggggattccgacagggattccggtggagggcaaaacgggaacaaccagaacggacagtcctccttgtctgtacgggaacaggatcggttcctgcccatcgcgaacgtgagccgaatcatgaagaaggcgttgccgtctaatgcaaaaatttcgaaggacgcgaaagagactgtgcaggagtgtgtgtccgagttcatcagcttcatcactggtgaggcgtcagataagtgccagagggagaagagaaagacgatcaacggtgacgacttgctgtgggccatgagtacacttggtttcgaagattacgtggagcctctgaaggtttacctacacaaataccgggagctagagggagagaagactggactagggaggccacagactggtggtgaggtcggagagcatcagagagatgctgtcggagatggcggtgggttctacggtggtggtggtgggatgcagtatcaccaacatcatcagtttcttcaccagcagaaccatatgtatggagccacaggtggcggtagcgacagtggaggtggagctgcctccggtaggacaaggacttaa

SEQ ID NO: 16 - protein, 190 aa, Artificial
  Synthetic Construct
MGDSDRDSGGGQNGNNQNGQSSLSVREQDRFLPIANVSRIMKKALPSNAKISKDAKETVQECVSEFISFITGEASDKCQREKRKTINGDDLLWAMSTLGFEDYVEPLKVYLHKYRELEGEKTGLGRPQTGGEVGEHQRDAVGDGGGFYGGGGGMQYHQHHQFLHQQNHMYGATGGGSDSGGGAASGRTRT

SEQ ID NO: 17 - DNA, 633 bp, Artificial
  synthetic chimeric sequence (NFYB-C6)
atgggggattccgacagggattccggtggagggcaaaacgggaacaaccagaacggacagtcctccttgtctgtacgggaacaggatcggttcctgcccatcgcgaacgtgagccgaatcatgaagaaggcgttgccgtctaatgcaaaaatttcgaaggacgcgaaagagactgtgcaggagtgtgtgtccgagttcatcagcttcatcactggtgaggcgtcagataagtgccagagggagaagagaaagacgatcaacggtgacgacttgctgtgggccatgagtacacttggtttcgaagattacgtggagcctctgaaggtttacctacacaaataccgggagctagagggagagaaggcttccacggccaagggtggtgaccagcaaggagggaaagaagggagtcaaggtgttatggggtccatgggtatgtcgggcggaatgaacggtatgaacggtacgatgaacgggaatatgcatggacatggaatcccggtgtcgatgcagatgctgcagcagtcgtacggacagcaggcacctccagggatgatgtattcccctcatcagatgatgccgcaataccagatgccaatgcagtctggtggaaaccagcctcgtggagtgtag

SEQ ID NO: 18 - protein, 210 aa, Artificial
  Synthetic Construct
MGDSDRDSGGGQNGNNQNGQSSLSVREQDRFLPIANVSRIMKKALPSNAKISKDAKETVQECVSEFISFITGEASDKCQREKRKTINGDDLLWAMSTLGFEDYVEPLKVYLHKYRELEGEKASTAKGGDQQGGKEGSQGVMGSMGMSGGMNGMNGTMNGNMHGHGIPVSMQMLQQSYGQQAPPGMMYSPHQMMPQYQMPMQSGGNQPRGV

SEQ ID NO: 19 - DNA, 678 bp, Artificial
  synthetic chimeric sequence (NFYB-C7)
atggccgatagctacggtcacaacgcaggttcaccagagagcagcccgcattctgataacgagtccgggggtcattaccgagaccaggatgctagcgtcagggagcaggacaggttcctgcccatcgccaacatcagtcgcatcatgaagaaggccatcccggctaacgggaagaccatcccggctaacgggaagatcgccaaggacgctaaggagaccgtgcaggagtgcgtctccgagttcatctccttcatcactagcgaagcgagtgacaagtgccagagggagaagcggaagaccatcaatggcgacgatctgctgtgggccatggccacgctggggtttgaagactacattgaacccctcaaggtgtacctgcagaagtacagagagatggagggtgatagcgcttccacggccaagggtggtgaccagcaaggagggaaagaagggagtcaaggtgttatggggtccatgggtatgtcgggcggaatgaacggtatgaacggtacgatgaacgggaatatgcatggacatggaatcccggtgtcgatgcagatgctgcagcagtcgtacggacagcaggcacctccagggatgatgtattcccctcatcagatgatgccgcaataccagatgccaatgcagtctggtggaaaccagcctcgtggagtgtag

SEQ ID NO: 20 - protein, 225 aa, Artificial
  Synthetic Construct
MADSYGHNAGSPESSPHSDNESGGHYRDQDASVREQDRFLPIANISRIMKKAIPANGKTIPANGKIAKDAKETVQECVSEFISFITSEASDKCQREKRKTINGDDLLWAMATLGFEDYIEPLKVYLQKYREMEGDSASTAKGGDQQGGKEGSQGVMGSMGMSGGMNGMNGTMNGNMHGHGIPVSMQMLQQSYGQQAPPGMMYSPHQMMPQYQMPMQSGGNQPRGV

SEQ ID NO: 21 - DNA, 549 bp, Artificial
  synthetic chimeric sequence (NFYB-C8)
atggccgatagctacggtcacaacgcaggttcaccagagagcagcccgcattctgataacgagtccgggggtcattaccgagaccaggatgcttctgtacgggaacaggatcggttcctgcccatcgcgaacgtgagccgaatcatgaagaaggcgttgccgtctaatgcaaaaatttcgaaggacgcgaaagagactgtgcaggagtgtgtgtccgagttcatcagcttcatcactggtgaggcgtcagataagtgccagagggagaagagaaagacgatcaacggtgacgacttgctgtgggccatgagtacacttggtttcgaagattacgtggagcctctgaaggtttacctacacaaataccgggagctagagggagagaagaagttaactgcaaaatctagcgatggctcaattaaaaaggatgcccttggtcatgtgggagcaagtagctcagctgcacaagggatgggccaacagggagcatacaaccaaggaatgggttatatgcaaccccagtaccataacggggatatctcaaactaa

SEQ ID NO: 22 - protein, 182 aa, Artificial
  Synthetic Construct
MADSYGHNAGSPESSPHSDNESGGHYRDQDASVREQDRFLPIANVSRIMKKALPSNAKISKDAKETVQECVSEFISFITGEASDKCQREKRKTINGDDLLWAMSTLGFEDYVEPLKVYLHKYRELEGEKKLTAKSSDGSIKKDALGHVGASSSAAQGMGQQGAYNQGMGYMQPQYHNGDISN

SEQ ID NO: 23 - DNA, 570 bp, Artificial
  synthetic chimeric sequence (NFYB-C9)
atggccgatagctacggtcacaacgcaggttcaccagagagcagcccgcattctgataacgagtccgggggtcattaccgagaccaggatgctagcgtcagggagcaggacaggttcctgcccatcgccaacatcagtcgcatcatgaagaaggccatcccggctaacgggaagaccatcccggctaacgggaagatcgccaaggacgctaaggagaccgtgcaggagtgcgtctccgagttcatctccttcatcactagcgaagcgagtgacaagtgccagagggagaagcggaagaccatcaatggcgacgatctgctgtgggccatggccacgctggggtttgaagactacattgaacccctcaaggtgtacctgcagaagtacagagagatggagggtgatagcaagttaactgcaaaatctagcgatggctcaattaaaaaggatgcccttggtcatgtgggagcaagtagctcagctgcacaagggatgggccaacagggagcatacaaccaaggaatgggttatatgcaaccccagtaccataacggggatatctcaaactaa

SEQ ID NO: 24 - protein, 189 aa, Artificial
  Synthetic Construct
MADSYGHNAGSPESSPHSDNESGGHYRDQDASVREQDRFLPIANISRIMKKAIPANGKTIPANGKIAKDAKETVQECVSEFISFITSEASDKCQREKRKTINGDDLLWAMATLGFEDYIEPLKVYLQKYREMEGDSKLTAKSSDGSIKKDALGHVGASSSAAQGMGQQGAYNQGMGYMQPQYHNGDISN

SEQ ID NO: 25 - DNA, 666 bp, Artificial
  synthetic chimeric sequence (NFYB-C10)
atggcggaagctccggcgagccctggcggcggcggcgggagccacgagagcgggagccccaggggaggcggaggcggtggcagcgtcagggagcaggacaggttcctgcccatcgccaacatcagtcgcatcatgaagaaggccatcccggctaacgggaagaccatcccggctaacgggaagatcgccaaggacgctaaggagaccgtgcaggagtgcgtctccgagttcatctccttcatcactagcgaagcgagtgacaagtgccagagggagaagcggaagaccatcaatggcgacgatctgctgtgggccatggccacgctggggtttgaagactacattgaacccctcaaggtgtacctgcagaagtacagagagatggagggtgatagcgcttccacggccaagggtggtgaccagcaaggagggaaagaagggagtcaaggtgttatggggtccatgggtatgtcgggcggaatgaacggtatgaacggtacgatgaacgggaatatgcatggacatggaatcccggtgtcgatgcagatgctgcagcagtcgtacggacagcaggcacctccagggatgatgtattcccctcatcagatgatgccgcaataccagatgccaatgcagtctggtggaaaccagcctcgtggagtgtag

SEQ ID NO: 26 - protein, 221 aa, Artificial
  Synthetic Construct
MAEAPASPGGGGGSHESGSPRGGGGGGSVREQDRFLPIANISRIMKKAIPANGKTIPANGKIAKDAKETVQECVSEFISFITSEASDKCQREKRKTINGDDLLWAMATLGFEDYIEPLKVYLQKYREMEGDSASTAKGGDQQGGKEGSQGVMGSMGMSGGMNGMNGTMNGNMHGHGIPVSMQMLQQSYGQQAPPGMMYSPHQMMPQYQMPMQSGGNQPRGV

SEQ ID NO: 27 - DNA, 537 bp, Artificial
  synthetic chimeric sequence (NFYB-C11)
atggcggaagctccggcgagccctggcggcggcggcgggagccacgagagcgggagccccaggggaggcggaggcggtggctctgtacgggaacaggatcggttcctgcccatcgcgaacgtgagccgaatcatgaagaaggcgttgccgtctaatgcaaaaatttcgaaggacgcgaaagagactgtgcaggagtgtgtgtccgagttcatcagcttcatcactggtgaggcgtcagataagtgccagagggagaagagaaagacgatcaacggtgacgacttgctgtgggccatgagtacacttggtttcgaagattacgtggagcctctgaaggtttacctacacaaataccgggagctagagggagagaagaagttaactgcaaaatctagcgatggctcaattaaaaaggatgcccttggtcatgtgggagcaagtagctcagctgcacaagggatgggccaacagggagcatacaaccaaggaatgggttatatgcaaccccagtaccataacggggatatctcaaactaa

SEQ ID NO: 28 - protein, 178 aa, Artificial
  Synthetic Construct
MAEAPASPGGGGGSHESGSPRGGGGGGSVREQDRFLPIANVSRIMKKALPSNAKISKDAKETVQECVSEFISFITGEASDKCQREKRKTINGDDLLWAMSTLGFEDYVEPLKVYLHKYRELEGEKKLTAKSSDGSIKKDALGHVGASSSAAQGMGQQGAYNQGMGYMQPQYHNGDISN

SEQ ID NO: 29 - DNA, 645 bp, Artificial
  synthetic chimeric sequence (NFYB-C12)
atggcggaagctccggcgagccctggcggcggcggcgggagccacgagagcgggagccccaggggaggcggaggcggtggctctgtacgggaacaggatcggttcctgcccatcgcgaacgtgagccgaatcatgaagaaggcgttgccgtctaatgcaaaaatttcgaaggacgcgaaagagactgtgcaggagtgtgtgtccgagttcatcagcttcatcactggtgaggcgtcagataagtgccagagggagaagagaaagacgatcaacggtgacgacttgctgtgggccatgagtacacttggtttcgaagattacgtggagcctctgaaggtttacctacacaaataccgggagctagagggagagaaggcttccacggccaagggtggtgaccagcaaggagggaaagaagggagtcaaggtgttatggggtccatgggtatgtcgggcggaatgaacggtatgaacggtacgatgaacgggaatatgcatggacatggaatcccggtgtcgatgcagatgctgcagcagtcgtacggacagcaggcacctccagggatgatgtattcccctcatcagatgatgccgcaataccagatgccaatgcagtctggtggaaaccagcctcgtggagtgtag

SEQ ID NO: 30 - protein, 214 aa, Artificial
  Synthetic Construct
MAEAPASPGGGGGSHESGSPRGGGGGGSVREQDRFLPIANVSRIMKKALPSNAKISKDAKETVQECVSEFISFITGEASDKCQREKRKTINGDDLLWAMSTLGFEDYVEPLKVYLHKYRELEGEKASTAKGGDQQGGKEGSQGVMGSMGMSGGMNGMNGTMNGNMHGHGIPVSMQMLQQSYGQQAPPGMMYSPHQMMPQYQMPMQSGGNQPRGV

SEQ ID NO: 31 - DNA, 657 bp, Physcomitrella patens
  CDS (1)..(657): CBF-A (EST69)
atggccgacagttacggccacaacgcaggttcacccgagagcagcccgcattctgataacgagtccggcggccattaccgtgatcaggacgcttctgtacgggagcaagaccggtttttgcccatcgcaaatgtgagccgaatcatgaagaaagcattgccatctaatgcgaagatatcgaaagacgccaaagagactgtgcaggagtgcgtatccgagttcatcagtttcattactggtgaggcgtccgacaagtgtcagagggaaaagaggaagacgatcaacggggatgacttgctgtgggccatgagtactcttggttttgaagattatgtggaacctctgaaggtgtacctacacaagtatcgtgaactggagggggagaaggcctctatggccaagggtggtgatcagcagggaggaaaagagagcaaccaaggaggtatggggtcgatgggcatggcaggcggaatcaacggcatgaacggaacgatgaacgggaacatgcatgggcatggaattcccgtatcgatgcagatgatgcaacagccgtacgcgcagcaggcacctccggggatgatatattctcctcatcaaatgatgccgcaataccagatgccgatgcagtctggtggaaaccaaccccgcggagtgtaa

SEQ ID NO: 32 - protein, 218 aa, Physcomitrella patens
MADSYGHNAGSPESSPHSDNESGGHYRDQDASVREQDRFLPIANVSRIMKKALPSNAKISKDAKETVQECVSEFISFITGEASDKCQREKRKTINGDDLLWAMSTLGFEDYVEPLKVYLHKYRELEGEKASMAKGGDQQGGKESNQGGMGSMGMAGGINGMNGTMNGNMHGHGIPVSMQMMQQPYAQQAPPGMIYSPHQMMPQYQMPMQSGGNQPRGV

SEQ ID NO: 33 - DNA, 453 bp, Zea mays
  CDS (1)..(453): NF-YB (ZM58019377)
atggccaacctcgtccgcctcatgcggcaggtgatccccaagggggtcaagatctcatcgcgcgccaaggacctcacgcacgactgcgccgtcgagttcgtcggcttcctcgccggcgaggcgtccgagctggccagggcgcagcaccgccgcaccatctcgcccgaggacttcacgcgctcgctccaggcgctcggcttcgacgactacgtcaggcccatgagcacctacatcagccgctaccgcgagcaggccaccagccccgccggctacagcggcgggttcgcgcggcgccctcctccccgtgcgacggcggcgccgtgcgtttccgatgaggaaacgcggagtcgtgttcccccacacggagagcacggtgatggctggcgcacgtcggtgaacacgcccgcgccgggcgggcacggctatgattacacttcgcctgataacatgtga

SEQ ID NO: 34 - protein, 150 aa, Zea mays
MANLVRLMRQVIPKGVKISSRAKDLTHDCAVEFVGFLAGEASELARAQHRRTISPEDFTRSLQALGFDDYVRPMSTYISRYREQATSPAGYSGGFARRPPPRATAAPCVSDEETRSRVPPHGEHGDGWRTSVNTPAPGGHGYDYTSPDNM

SEQ ID NO: 35 - DNA, 570 bp, Zea mays
  CDS (1)..(570): NF-YB (ZM61020893)
atgggccgcaaggcgaagcgatgtgctgggaagaaggacgacggccgtgaagagaaggttgccgccgcgccggcggccgagggcgcgtcgtcgtcgtcgtcggacggggaaggagcaggcaccgcggccaccgggctgccgatggccaacctcgtccgcctcatgcggcaggtgatccccaagggggtcaagatctcatcgcgcgccaaggacctcacgcacgactgcgccatcgagttcgtcggcttcctcgccggcgaggcgtccgagctggccagggcgcagcaccgccgcaccatctcgcccgaggacttcacgcgctcgctccaggcgctcggcttcgacgactacgtcaggcccatgagcacctacatcagccgctaccgcgagcaggccaccagccccgccggctacagcggcttcgcgcggcgccctcctccccgtgcggcggcggcgccgtgcgtttccgatgaggaaacgcggagtcgtgttcccccacacggagagttcggtgatggcaggagcacgtcggtgaacacgcccgcgccgggcgggcatggctatatatga

SEQ ID NO: 36 - protein, 189 aa, Zea mays
MGRKAKRCAGKKDDGREEKVAAAPAAEGASSSSSDGEGAGTAATGLPMANLVRLMRQVIPKGVKISSRAKDLTHDCAIEFVGFLAGEASELARAQHRRTISPEDFTRSLQALGFDDYVRPMSTYISRYREQATSPAGYSGFARRPPPRAAAAPCVSDEETRSRVPPHGEFGDGRSTSVNTPAPGGHGYI

SEQ ID NO: 37 - DNA, 906 bp, Zea mays
  CDS (1)..(906): NF-YB (ZM62014459)
atggacccgatggacatcgtcggcaagtccaaggaggacgtctccctccccaaatcaacaatgtttaagataattaaggagatgcttcctcctgatgtacgagtggcaagagatgcacaggatcttcttgttgaatgctgtgtagagttcatcaatctcctttcgtctgaatcgaatgaagtgtgcagcagagaagagaaaaaaacgattgctcctgagcatgttatcaaggctctaagtgatcttggcttcagagagtacattgaggaggtttatgctgcatatgaacagcacaaacttgatactttggattctccaaaagctggcaaattcactgggattgagatgacagaggaagaagctgttgctgagcaacaaaggatgtttgctgaggctcgagcaaggatgaataatggagcccccaaaccaaaggagacagaacaagagccgccacagcaaccacaagcacaaccccaacttcagctgcatactgaaccacagcaacccgtgcaatctcaagtgcagttgcattcgccaacacagcattccctccaacctcaagtgcagctgcacccccagccacaacagctcccacaagtgcaggtgcactctcagacgcagctgcaccctcagccacagcagccccaagtgcaggtccatccgcagctgcagcaactcccacaattacaggcccactcgcagccaccgcaaccccaagtgcaaatccacccacagccgcagcagcccccacaagtgcagttgcagtcatcagtccagcagacctcccaaccccaacctcaggtacatctctacaatcacagggggggctcacaagcgcagctgcagccccaactgccaggtcagctgcagacacaggggcaaaccggacctggcattgacagttag

SEQ ID NO: 38 - protein, 301 aa, Zea mays
MDPMDIVGKSKEDVSLPKSTMFKIIKEMLPPDVRVARDAQDLLVECCVEFINLLSSESNEVCSREEKKTIAPEHVIKALSDLGFREYIEEVYAAYEQHKLDTLDSPKAGKFTGIEMTEEEAVAEQQRMFAEARARMNNGAPKPKETEQEPPQQPQAQPQLQLHTEPQQPVQSQVQLHSPTQHSLQPQVQLHPQPQQLPQVQVHSQTQLHPQPQQPQVQVHPQLQQLPQLQAHSQPPQPQVQIHPQPQQPPQVQLQSSVQQTSQPQPQVHLYNHRGGSQAQLQPQLPGQLQTQGQTGPGIDS

SEQ ID NO: 39 - DNA, 528 bp, Zea mays
  CDS (1)..(528): NF-YB (ZM62260706)
atgccacgtaagatcaccttggtcaaggagggggagccgcagccggtggacgctactcaaggggagcccgctccggcaccggcggcaccgacggccgcgttgacggaggcggaggtggacgagctgcccagggccatcgtgcgccgccttgtcaaggacaagctttcccacgtcgccggtggggaaggagccgaggtcatcgtcaacaaggacgccatggccgcgttcgccgagagcgcccgcatcttcatccactatctctccgccactgccaatgatatgtgcaaggaatcaaagaggcagaccatcaacgctgatgatgtcctcaatgctcttgatgacatggagttttcggagtttgtagagccccttaggacttcactgcaagagtttagaaacaaaaatgcagacaaaaggtcagaagcaaagaaacaacagaaggaaaagagaaggaaattaaatgaagagacacttccgcagaaagaaaatgatccagctgatgatgccaaggagaatggagtagcttaa

SEQ ID NO: 40 - protein, 175 aa, Zea mays
MPRKITLVKEGEPQPVDATQGEPAPAPAAPTAALTEAEVDELPRAIVRRLVKDKLSHVAGGEGAEVIVNKDAMAAFAESARIFIHYLSATANDMCKESKRQTINADDVLNALDDMEFSEFVEPLRTSLQEFRNKNADKRSEAKKQQKEKRRKLNEETLPQKENDPADDAKENGVA

SEQ ID NO: 41 - DNA, 795 bp, Zea mays
  CDS (1)..(795): NF-YB (ZM59456239)
atggactccagcttcctccctgccggcgcggacaatggctcggcgggcggcgccaacaatggcggcggcgctgctcagcaggcgccgccgatccgcgagcaggaccggctgatgccgatcgcgaacgtcatccgcatcatgcggcgcgtgctgccggcgcacgccaagatctcggacgacgccaaggagacgatccaggagtgcgtgtcggagtacatcagcttcatcacgggggaggccaacgagcggtgccagcgggagcagcgcaagaccatcaccgccgaggacgtgctgtgggccatgagccgcctcggcttcgacgactacgtcgagccgctcagcgtctacctccaccgctaccgcgagttcgagggcgaggcgcggggcgtcggcctcgccccggcccctccgcgcggcgaccaccaccaccaccaccactccgtgccgccatcgatgctcaacaagtcccgcgggcccggctccggagccgtcatgctaccgcaccaccaccaccacgacatgcacgcctccatgtacgggggcgccgtgcccccgccgccgcaccacggcttcctcatgccacacccacagggcggccactacctgccttacccctacgagcccacgtcgtacggcggcgagcacgccttggccagcgggtactatggaggggccgcgtacgcgccgggcaacaacggcgggagcggcgatggcagcggcgggagcgcgtcgcacgcaccgccgggcggcagcggcggcggcttcgaccacccgcacacgttcgcgtacaagtag

SEQ ID NO: 42 - protein, 264 aa, Zea mays
MDSSFLPAGADNGSAGGANNGGGAAQQAPPIREQDRLMPIANVIRIMRRVLPAHAKISDDAKETIQECVSEYISFITGEANERCQREQRKTITAEDVLWAMSRLGFDDYVEPLSVYLHRYREFEGEARGVGLAPAPPRGDHHHHHHSVPPSMLNKSRGPGSGAVMLPHHHHHDMHASMYGGAVPPPPHHGFLMPHPQGGHYLPYPYEPTSYGGEHALASGYYGGAAYAPGNNGGSGDGSGGSASHAPPGGSGGGFDHPHTFAYK

SEQ ID NO: 43 - DNA, 786 bp, Zea mays
  CDS (1)..(786): NF-YB (ZM59473285)
atgaataatccccaaaaccctaaagccagtgctccttgcaccttgccaccggagcttcccaaagaagcagtggcgaccgacgaagcaccgccgccaatgggcaacaacaacaacacggaatcggcgaccgcgacgatggtccgggagcaggaccggctgatgcccgtggccaacgtgtcccgcatcatgcgccaagtgctgcctccgtacgccaagatctccgacgacgccaaggaggtgatccaggagtgcgtgtcggagttcatcagcttcgtcactggcgaggcgaacgagcggtgccacaccgagcgccgcaagaccgtcacctccgaggacatcgtgtgggccatgagccgcctcggcttcgacgactacgtcgcgcccctcggcgccttcctccagcgcatgcgcgacagcgaccacggcggtgaagagcgcggcggccctgcagggcgtggtggctcgcgccgcggctcgtcgtccttgccgctccactgcccgcagcagatgcaccacctgcacccagccgtctgccggcgtccgcaccagagcgtgtcgcctgctgcaggatacgccgtccggcccgttccccgcccgatgccagccagtgggtaccgcatgcagggcggagaccaccgcagcgtgggcggcgtggctccctgcagctacggaggggcgctcgtccaggccggtggaacccaacacgttgttggattccacgacgacgaggcaagctcttcgagtgaaaatccgccgccggaggggcgtgccgctggctcgaactag

SEQ ID NO: 44 - protein, 261 aa, Zea mays
MNNPQNPKASAPCTLPPELPKEAVATDEAPPPMGNNNNTESATATMVREQDRLMPVANVSRIMRQVLPPYAKISDDAKEVIQECVSEFISFVTGEANERCHTERRKTVTSEDIVWAMSRLGFDDYVAPLGAFLQRMRDSDHGGEERGGPAGRGGSRRGSSSLPLHCPQQMHHLHPAVCRRPHQSVSPAAGYAVRPVPRPMPASGYRMQGGDHRSVGGVAPCSYGGALVQAGGTQHVVGFHDDEASSSSENPPPEGRAAGSN

SEQ ID NO: 45 - DNA, 537 bp, Zea mays
  CDS (1)..(537): NF-YB (ZM59153552)
atggcggaagctccggcgagccctggcggcggcggcgggagccacgagagcgggagccccaggggaggcggaggcggtggcagcgtcagggagcaggacaggttcctgcccatcgccaacatcagtcgcatcatgaagaaggccatcccggctaacgggaagatcgccaaggacgctaaggagaccgtgcaggagtgcgtctccgagttcatctccttcatcactagcgaagcgagtgacaagtgccagagggagaagcggaagaccatcaatggcgacgatctgctgtgggccatggccacgctggggtttgaagactacattgaacccctcaaggtgtacctgcagaagtacagagagatggagggtgatagcaagttaactgcaaaatctagcgatggctcaattaaaaaggatgcccttggtcatgtgggagcaagtagctcagctgcacaagggatgggccaacagggagcatacaaccaaggaatgggttatatgcaaccccagtaccataacggggatatctcaaactaa

SEQ ID NO: 46 - protein, 178 aa, Zea mays
MAEAPASPGGGGGSHESGSPRGGGGGGSVREQDRFLPIANISRIMKKAIPANGKIAKDAKETVQECVSEFISFITSEASDKCQREKRKTINGDDLLWAMATLGFEDYIEPLKVYLQKYREMEGDSKLTAKSSDGSIKKDALGHVGASSSAAQGMGQQGAYNQGMGYMQPQYHNGDISN

SEQ ID NO: 47 - DNA, 543 bp, Zea mays
  CDS (1)..(543): NF-YB (ZM62055702)
atggcggacgctccggcgagccctgggggcggaggcgggagccacgagagcgggagccccaggggcggcggaggtggaggcggtggcagcgtcagggagcaggacaggttcctgcccatcgccaacatcagtcgcatcatgaagaaggccatcccggctaacgggaagatcgccaaggacgctaaggagaccgtgcaggagtgcgtctcggagttcatctccttcatcactagcgaggcgagtgacaagtgccagagggagaagcggaagaccatcaatggcgacgacctgctgtgggccatggccacgctggggtttgaggactatattgaacccctcaaggtgtacctgcagaagtacagagagatggagggtgatagtaagttaacttcaaaatccagcgatggctccattaaaaaggatgcccttggtcatgtgggagcaagtagctcagctgtacaagggatgggtcagcaaggaacatacaaccaaggaatgggttatatgcaacctcagtaccataacggagatatctcgaactaa

SEQ ID NO: 48 - protein, 180 aa, Zea mays
MADAPASPGGGGGSHESGSPRGGGGGGGGSVREQDRFLPIANISRIMKKAIPANGKIAKDAKETVQECVSEFISFITSEASDKCQREKRKTINGDDLLWAMATLGFEDYIEPLKVYLQKYREMEGDSKLTSKSSDGSIKKDALGHVGASSSAVQGMGQQGTYNQGMGYMQPQYHNGDISN

SEQ ID NO: 49 - DNA, 495 bp, Zea mays
  CDS (1)..(495): NF-YB (ZM67286704)
atggccgacgacggcgggagccacgagggcagcggcggcggcggaggcgtccgggagcaggaccggttcctgcccatcgccaacatcagccggatcatgaagaaggccgtcccggccaacggcaagatcgccaaggacgctaaggagaccctgcaggagtgcgtctccgagttcatatcattcgtgaccagcgaggccagcgacaaatgccagaaggagaaacgaaagacaatcaacggggacgatttgctctgggcgatggccactttaggattcgaggagtacgtcgagcctctcaagatttacctacaaaagtacaaagagatggagggtgatagcaagctgtctacaaaggctggcgagggctctgtaaagaaggatgcaattagtccccatggtggcaccagtagctcaagtaatcagttggttcagcatggagtctacaaccaagggatgggctatatgcagccacagtaccacaatggggaaacctaa

SEQ ID NO: 50 - protein, 164 aa, Zea mays
MADDGGSHEGSGGGGGVREQDRFLPIANISRIMKKAVPANGKIAKDAKETLQECVSEFISFVTSEASDKCQKEKRKTINGDDLLWAMATLGFEEYVEPLKIYLQKYKEMEGDSKLSTKAGEGSVKKDAISPHGGTSSSSNQLVQHGVYNQGMGYMQPQYHNGET

SEQ ID NO: 51 - DNA, 498 bp, Zea mays
  CDS (1)..(498): NF-YB (ZM65405678)
atgaagaaggcgctcccggccaacgccaagatctccaaggacgccaaggagacggtgcaggagtgcgtgtcggagttcatctccttcatcaccggcgaggcctccgacaagtgccagcgcgagaagcgcaagaccatcaacggcgacgacctgctctgggccatgaccacgctcggcttcgaggactacgtcgagccgctcaagctctacctccacaagttccgcgagctcgagggcgagaaggcggccacgacgagcgcctcctccggcccgcagccgccgctgcacagggagacgacgccgtcgtcgtcaacgcacaatggcgcgggcgggggaagcggtatgatcatgatgatgggacagcccatgtacggcggctccccgccggccgcgtcgtccgggtcgtacccgcaccaccagatggccatgggcggaaaaggtggcgcctatggctacggcggaagctcgtcgtcgtcgccgtcagggctcggcaggtag

SEQ ID NO: 52 - protein, 165 aa, Zea mays
MKKALPANAKISKDAKETVQECVSEFISFITGEASDKCQREKRKTINGDDLLWAMTTLGFEDYVEPLKLYLHKFRELEGEKAATTSASSGPQPPLHRETTPSSSTHNGAGGGSGMIMMMGQPMYGGSPPAASSGSYPHHQMAMGGKGGAYGYGGSSSSSPSGLGR

SEQ ID NO: 53 - DNA, 885 bp, Zea mays
  CDS (1)..(885): NF-YB (ZM62083966)
atggacatcgtggggaagtctaaggaggacgtttccctccccaaatcaacaatggttaagataattaaggagatgcttcctcctgatgtacgagtggcaagagatgcacaggatcttcttgttgaatgctgcgtagagttcatcaatctcctttcgtctgaatccaatgaagtgtgcagcagagaagagaagaaaacaattgctcctgagcatgttatcaaggctctaagtgatcttggcttcagagagtacattgaggaggtttatgctgcgtatgaacagcacaaactcgagactctggattctccaaaagctggcaagttcactaggattgagatgacagaggaagaagctgttgctgagcaacaaaggatgtttgctgaggctcgagcaaggatgaataacggagcccccaagccaaaggagccagaacaagaaccgccacagctaccacaagcacaaccccaacttcagctgcatactgaaccacagcaacccatgcaatctcaagttcagttgcattcgcaaacacaacactacctccaacctcaactgcagctccaccatcagccacaacagctcccacaagtgcagctgcactctcagccgcagctgcaaccccaagtgcatctgcaccctcagccgcagttgcccccccaattgcaggtccatcagcagctgcagcaacccccccaagtgcaggtcccccagcagcccgaagtgcagccccaagaagcgcagttgcaatcatcagcccagcaaacctcccaaccgcaacctcaggcacaactacaatcacaggggcactcacaagcgcagctgcaagccggactgctgggtcagttgcagacacaggggcaaaccggacctgacatggatagttag

SEQ ID NO: 54 - protein, 294 aa, Zea mays
MDIVGKSKEDVSLPKSTMVKIIKEMLPPDVRVARDAQDLLVECCVEFINLLSSESNEVCSREEKKTIAPEHVIKALSDLGFREYIEEVYAAYEQHKLETLDSPKAGKFTRIEMTEEEAVAEQQRMFAEARARMNNGAPKPKEPEQEPPQLPQAQPQLQLHTEPQQPMQSQVQLHSQTQHYLQPQLQLHHQPQQLPQVQLHSQPQLQPQVHLHPQPQLPPQLQVHQQLQQPPQVQVPQQPEVQPQEAQLQSSAQQTSQPQPQAQLQSQGHSQAQLQAGLLGQLQTQGQTGPDMDS

SEQ ID NO: 55 - DNA, 495 bp, Zea mays
  CDS (1)..(495): NF-YB (ZmEvi061009.1)
atggcggaagctccggcgagccctggcggcggcggcgggagccacgagagcgggagccccaggggaggcggaggcggtggcagcgtcagggagcaggacaggttcctgcccatcgccaacatcagtcgcatcatgaagaaggccatcccggctaacgggaagatcgccaaggacgctaaggagaccgtgcaggagtgcgtctccgagttcatctccttcatcactagcgaggcgagtgacaagtgccagagggagaagcggaagaccatcaatggcgacgatctgctgtgggccatggccacgctggggtttgaagactacattgaacccctcaaggtgtacctgcagaagtacagagaggtgcagggtgatagcaagttaactgcaaaatctagcgatggctcaattaaaaaggatgcccttggtcatgtgggagcaagtagctcagctgcacaaggggttaagttttcttggactgttccgttattcttgtga

SEQ ID NO: 56 - protein, 164 aa, Zea mays
MAEAPASPGGGGGSHESGSPRGGGGGGSVREQDRFLPIANISRIMKKAIPANGKIAKDAKETVQECVSEFISFITSEASDKCQREKRKTINGDDLLWAMATLGFEDYIEPLKVYLQKYREVQGDSKLTAKSSDGSIKKDALGHVGASSSAAQGVKFSWTVPLFL

SEQ ID NO: 57 - protein, 178 aa, Zea mays
  MISC_FEATURE (1)..(178): NF-YB (ZmEvi061009.2)
MAEAPASPGGGGGSHESGSPRGGGGGGSVREQDRFLPIANISRIMKKAIPANGKIAKDAKETVQECVSEFISFITSEASDKCQREKRKTINGDDLLWAMATLGFEDYIEPLKVYLQKYREQQGDSKLTAKSSDGSIKKDALGHVGASSSAAQGMGQQGAYNQGMGYMQPQYHNGDISN

SEQ ID NO: 58 - protein, 174 aa, Zea mays
  MISC_FEATURE (1)..(174): NF-YB (ZmEvi061009.3)
MAEAPASPGGGGGSHESGSPRGGGGGGSVREQDRFLPIANISRIMKKAIPANGKIAKDAKETVQECVSEFISFITSEASDKCQREKRKTINGDDLLWAMATLGFEDYIEPLKVYLQKYREVQGDSKLTAKSSDGSIKKDALGHVGASSSAAQGGAYNQGMGYMQPQYHNGDISN

SEQ ID NO: 59 - DNA, 450 bp, Zea mays
  CDS (1)..(450): NF-YB (AC198485)
atggcggaagctccggcgagccctggcggcggcggcgggagccacgagagcgggagccccaggggaggcggaggcggtggcagcgtcagggagcaggacaggttcctgcccatcgccaacatcagtcgcatcatgaagaaggccatcccggctaacgggaagatcgccaaggacgctaaggagaccgtgcaggagtgcgtctccgagttcatctccttcatcactagcgaggcgagtgacaagtgccagagggagaagcggaagaccatcaatggcgacgatctgctgtgggccatggccacgctggggtttgaagactacattgaacccctcaaggtgtacctgcagaagtacagagaggtgcgtacggtgtttgggaatttgggggtcaggtcgtgcaatcgccaatctgtcaccttgtcgatcgtacctctgattgaacttaaataa

SEQ ID NO: 60 - protein, 149 aa, Zea mays
MAEAPASPGGGGGSHESGSPRGGGGGGSVREQDRFLPIANISRIMKKAIPANGKIAKDAKETVQECVSEFISFITSEASDKCQREKRKTINGDDLLWAMATLGFEDYIEPLKVYLQKYREVRTVFGNLGVRSCNRQSVTLSIVPLIELK

SEQ ID NO: 61 - DNA, 465 bp, Zea mays
  CDS (1)..(465): NF-YB (AC203785)
atggcggacgctccggcgagccctgggggcggaggcgggagccacgagagcgggagccccaggggcggcggaggtggaggcggtggcagcgtcagggagcaggacaggttcctgcccatcgccaacatcagtcgcatcatgaagaaggccatcccggctaacgggaagatcgccaaggacgctaaggagaccgtgcaggagtgcgtctcggagttcatctccttcatcactagggcgagtgacaagtgccagagggagaagcggaagaccatcaatggcgacgacctgctgtgggccatggccacgctggggtttgaggactatattgaacccctcaaggtgtacctgcagaagtacagagaggtgcgtactgatgaaggtattggggacttcaggttaattggattgatttggtggtcaggttgtgcaatcaccaatctgtcaccttgtcgattgtacctctga

SEQ ID NO: 62 - protein, 154 aa, Zea mays
MADAPASPGGGGGSHESGSPRGGGGGGGGSVREQDRFLPIANISRIMKKAIPANGKIAKDAKETVQECVSEFISFITRASDKCQREKRKTINGDDLLWAMATLGFEDYIEPLKVYLQKYREVRTDEGIGDFRLIGLIWWSGCAITNLSPCRLYL

SEQ ID NO: 63 - DNA, 570 bp, Zea mays
  CDS (1)..(570): NF-YB (AC210260)
gctgggaagaaggacgacggccgtgaagagaaggttgccgccgcgccggcggccgagggcgcgtcgtcgtcgtcgtcggacggggaaggagcaggcaccgcggccaccgggctgccgatggccaacctcgtccgcctcatgcggcaggtgatccccaagggggtcaagatctcatcgcgcgccaaggacctcacgcacgactgcgccgtcgagttcgtcggcttcctcgccggcgaggcgtccgagctggccagggcgcagcaccgccgcaccatctcgcccgaggacttcacgcgctcgctccaggcgctcggcttcgacgactacgtcaggcccatgagcacctacatcagccgctaccgcgagcaggccaccagccccgccggctacagcggcgggttcgcgcggcgccctcctccccgtgcgacggcggcgccgtgcgtttccgatgaggaaacgcggagtcgtgttcccccacacggagagcacggtgatggctggcgcacgtcggtgaacacgcccgcgccgggcgggcacggctatgattacacttcgcctgataacatgtga

SEQ ID NO: 64 - protein, 189 aa, Zea mays
AGKKDDGREEKVAAAPAAEGASSSSSDGEGAGTAATGLPMANLVRLMRQVIPKGVKISSRAKDLTHDCAVEFVGFLAGEASELARAQHRRTISPEDFTRSLQALGFDDYVRPMSTYISRYREQATSPAGYSGGFARRPPPRATAAPCVSDEETRSRVPPHGEHGDGWRTSVNTPAPGGHGYDYTSPDNM

SEQ ID NO: 65 - DNA, 843 bp, Zea mays
  CDS (1)..(843): NF-YB (AC205600)
atgtttaagataattaaggagatgcttcctcctgatgtacgagtggcaagagatgcacaggatcttcttgttgaatgctgtgagttcatcaatctcctttcgtctgaatcgaatgaagtgtgcagcagagaagagaaaaaaacgattgctcctgagcatgttatcaaggctctaagtgatcttggcttcagagagtacattgaggaggtttatgctgcatatgaacagcacaaacttgatactttggattctccaaaagctggcaaattcactgggattgagatgacagaggaagaagctgttgctgagcaacaaaggatgtttgctgaggctcgagcaaggatgaataatggagcccccaaaccaaaggagacagaacaagagccgccacagcaaccacaagcacaaccccaacttcagctgcatactgaaccacagcaacccgtgcaatctcaagtgcagttgcattcgccaacacagcattccctccaacctcaagtgcagctgcacccccagccacaacagctcccacaagtgcaggtgcactctcagacgcagctgcaccctcagccacagcagccccaagtgcaggtccatccgcagctgcagcaactcccacaattacaggcccactcgcagccaccgcaaccccaagtgcaaatccacccacagccgcagcagcccccacaagtgcagttgcagtcatcagtccagcagacctcccaaccccaacctcaggtacatctctacaatcacagggggggctcacaagcgcagctgcagccccaactgccaggtcagctgcagacacaggggcaaaccggacctggcattgacagttag

SEQ ID NO: 66 - protein, 280 aa, Zea mays
MFKIIKEMLPPDVRVARDAQDLLVECCEFINLLSSESNEVCSREEKKTIAPEHVIKALSDLGFREYIEEVYAAYEQHKLDTLDSPKAGKFTGIEMTEEEAVAEQQRMFAEARARMNNGAPKPKETEQEPPQQPQAQPQLQLHTEPQQPVQSQVQLHSPTQHSLQPQVQLHPQPQQLPQVQVHSQTQLHPQPQQPQVQVHPQLQQLPQLQAHSQPPQPQVQIHPQPQQPPQVQLQSSVQQTSQPQPQVHLYNHRGGSQAQLQPQLPGQLQTQGQTGPGIDS

SEQ ID NO: 67 - DNA, 894 bp, Zea mays
  CDS (1)..(894): NF-YB (ZmEvi005132)
atggacccgatggacatcgtggggaagtctaaggaggacgtttccctccccaaagcaacaatggttaagataattaaggagatgcttcctcctgatgtacgagtggcaagagatgcacaggatcttcttgttgaatgctgcgtagagttcatcaatctcctttcgtctgaatccaatgaagtgtgcagcagagaagagaagaaaacaattgctcctgagcatgttatcaaggctctaagtgatcttggcttcagagagtacattgaggaggtttatgctgcgtatgaacagcacaaactcgagactctggattctccaaaagctggcaagttcactaggattgagatgacagaggaagaagctgttgctgagcaacaaaggatgtttgctgaggctcgagcaaggatgaataacggagcccccaagccaaaggagccagaacaagaaccgccacagctaccacaagcacaaccccaacttcagctgcatactgaaccacagcaacccatgcaatctcaagttcagttgcattcgcaaacacaacactacctccaacctcaactgcagctccaccatcagccacaacagctcccacaagtgcagctgcactctcagccgcagctgcaaccccaagtgcatctgcaccctcagccgcagttgcccccacaattgcaggtccatcagcagctgcagcaacccccacaagtgcaggtccaccagcagcccgaagtgcagccccaagaagcgcagttgcaatcatcagcccagcaaacctcccaaccgcaacctcaggcacaactacaatcacaggggcactcacaagcgcagctgcaagccggactgctgggtcagttgcagacacaggcgcaaaccggacctgacatggatagttaa

SEQ ID NO: 68 - protein, 297 aa, Zea mays
MDPMDIVGKSKEDVSLPKATMVKIIKEMLPPDVRVARDAQDLLVECCVEFINLLSSESNEVCSREEKKTIAPEHVIKALSDLGFREYIEEVYAAYEQHKLETLDSPKAGKFTRIEMTEEEAVAEQQRMFAEARARMNNGAPKPKEPEQEPPQLPQAQPQLQLHTEPQQPMQSQVQLHSQTQHYLQPQLQLHHQPQQLPQVQLHSQPQLQPQVHLHPQPQLPPQLQVHQQLQQPPQVQVHQQPEVQPQEAQLQSSAQQTSQPQPQAQLQSQGHSQAQLQAGLLGQLQTQAQTGPDMDS

SEQ ID NO: 69 - DNA, 525 bp, Zea mays
  CDS (1)..(525): NF-YB (AC203676)
atgccacgtaagatcaccttggtcaaggagggggagccgcagccggtggacgctgctcaaggggagcccgctccggcaccggcggcaccgacggccgcgttgacggaggcggaggtggacgagctgcccagggccatcgtgcgccgccttgtcaaggacaagctttcccacgtcgccggtggggaaggagccgaggtcatcgtcaacaaggacgccatggccgcgttcgccgagagcgcccgcatcttcatccactatctctccgccgccaatgatatgtgcaaggaatcaaagaggcagaccatcaacgctgatgatgtcctcaatgctcttgatgacatggagttttcggagtttgtagagccccttaggacttcactgcaagagtttagaaacaaaaatgcagacaaaaggtcagaagcaaagaaacaacagaaggaaaagagaaggaaattaaatgaagagacacttccgcagaaagaaaatgatccagctgatgatgccaaggagaatggagtagcttaa

SEQ ID NO: 70 - protein, 174 aa, Zea mays
MPRKITLVKEGEPQPVDAAQGEPAPAPAAPTAALTEAEVDELPRAIVRRLVKDKLSHVAGGEGAEVIVNKDAMAAFAESARIFIHYLSAANDMCKESKRQTINADDVLNALDDMEFSEFVEPLRTSLQEFRNKNADKRSEAKKQQKEKRRKLNEETLPQKENDPADDAKENGVA

SEQ ID NO: 71 - DNA, 789 bp, Zea mays
  CDS (1)..(789): NF-YB (AC188837)
atgaataatccccaaaaccctaaagccagtgctccttgcaccttgccaccggagcttcccaaagaagcagtggcgaccgacgaagcaccgccgccaatgggcaacaacaacaacacggaatcggcgacggcgacgatggtccgggagcaggaccggctgatgcccgtggccaacgtgtcccgcatcatgcgccaagtgctgcctccgtacgccaagatctccgacgacgccaaggaggtgatccaggagtgcgtgtcggagttcatcagcttcgtcactggcgaggcgaacgagcggtgccacaccgagcgccgcaagaccgtcacctccgaggacatcgtgtgggccatgagccgcctcggcttcgacgactacgtcgcgcccctcggcgccttcctccagcgcatgcgcgacgacagcgaccacggcggtgaagagcgcggcggccctgcagggcgtggtggctcgcgccgcggctcgtcgtccttgccgctccactgcccgcagcagatgcaccacctgcacccagccgtctgccggcgtccgcaccagagcgtgtcgcctgctgcaggatacgccgtccggcccgttccccgcccgatgccagccagtgggtaccgcatgcagggcggagaccaccgcagcgtgggcggcgtggctccctgcagctacggaggggcgctcgtccaggccggtggaacccaacacgttgttggattccacgacgacgaggcaagctcttcgagtgaaaatccgccgccggaggggcgtgccgctggctcgaactag

SEQ ID NO: 72 - protein, 262 aa, Zea mays
MNNPQNPKASAPCTLPPELPKEAVATDEAPPPMGNNNNTESATATMVREQDRLMPVANVSRIMRQVLPPYAKISDDAKEVIQECVSEFISFVTGEANERCHTERRKTVTSEDIVWAMSRLGFDDYVAPLGAFLQRMRDDSDHGGEERGGPAGRGGSRRGSSSLPLHCPQQMHHLHPAVCRRPHQSVSPAAGYAVRPVPRPMPASGYRMQGGDHRSVGGVAPCSYGGALVQAGGTQHVVGFHDDEASSSSENPPPEGRAAGSN

SEQ ID NO: 73 - DNA, 564 bp, Zea mays
  CDS (1)..(564): NF-YB (AC203033)
gccgacgacggcgggagccacgagggcagcggcggcggcggaggcgtccgggagcaggaccggttcctgcccatcgccaacatcagccggatcatgaagaaggccgtcccggccaacggcaagatcgccaaggacgctaaggagaccctgcaggagtgcgtctccgagttcatatcattcgtgaccagcgaggccagcgacaaatgccagaaggagaaacgaaagacaatcaacggggacgatttgctctgggcgatggccactttaggattcgaggagtacgtcgagcctctcaagatttacctacaaaagtacaaagagggtgatagcaagctgtctacaaaggctggcgagggctctgtaaagaaggatgcaattagtccccatggtggcaccagtagctcaagtaatcagttggttcagcatggagtctacaaccaagggatgggctatatgcagccacagctagggaagtctctgcattgcttaccatgtgtattggcagaaaacaggaggcacttacaaagggtgttaatctctgcgatggctgcctctcaggtgtaa

SEQ ID NO: 74 - protein, 187 aa, Zea mays
ADDGGSHEGSGGGGGVREQDRFLPIANISRIMKKAVPANGKIAKDAKETLQECVSEFISFVTSEASDKCQKEKRKTINGDDLLWAMATLGFEEYVEPLKIYLQKYKEGDSKLSTKAGEGSVKKDAISPHGGTSSSSNQLVQHGVYNQGMGYMQPQLGKSLHCLPCVLAENRRHLQRVLISAMAASQV

SEQ ID NO: 75 - DNA, 639 bp, Zea mays
  CDS (1)..(639): NF-YB (AC187072)
atgccggactccgacaacgagtccggcgggccgagcaacgcggagttctcgtcgccgcgggagcaggaccggttcctgccgatcgcgaacgtgagccggatcatgaagaaggcgctcccggccaacgccaagatctccaaggacgccaaggagacggtgcaggagtgcgtgtcggagttcatctccttcatcaccggcgaggcctccgacaagtgccagcgcgagaagcgcaagaccatcaacggcgacgacctactctgggccatgaccacgctcggcttcgaggactacgtcgagccgctcaagctctacctccacaagttccgcgagctcgagggcgagaaggcggccacgacgagcgcctcctccggcccgcagccgccgctgcacagggagacgacgccgtcgtcgtcaacgcacaatggcgcgggcgggcccgtcgggggatacggcatgtacggcggcgcgggcgggggaagcggtatgatcatgatgatgggacagcccatgtacggcggctccccgccggccgcgtcgtccgggtcgtacccgcaccaccagatggccatgggcggaaaaggtggcgcctatggctacggcggaggctcgtcgtcgtcgccgtcagggctcggcaggtag

SEQ ID NO: 76 - protein, 212 aa, Zea mays
MPDSDNESGGPSNAEFSSPREQDRFLPIANVSRIMKKALPANAKISKDAKETVQECVSEFISFITGEASDKCQREKRKTINGDDLLWAMTTLGFEDYVEPLKLYLHKFRELEGEKAATTSASSGPQPPLHRETTPSSSTHNGAGGPVGGYGMYGGAGGGSGMIMMMGQPMYGGSPPAASSGSYPHHQMAMGGKGGAYGYGGGSSSSPSGLGR

SEQ ID NO: 77 - DNA, 639 bp, Zea mays; identical to SEQ ID NO: 75
  CDS (1)..(639): NF-YB (ZmEvi013724)

SEQ ID NO: 78 - protein, 212 aa, Zea mays; identical to SEQ ID NO: 76

SEQ ID NO: 79 - DNA, 354 bp, Zea mays
  CDS (1)..(354): NF-YB (AC204839)
atgccggactccgacaacgacgagtccggcgggccgagcaacgcggacttctcgtcgccgcgggagcaggaccggttcctgccgatcgcgaacgtgagccggatcatgaagaaggcgctcccggccaacgccaagatatccaaggacggcaaggagacggtgcaggagtgcgtgtccgagttcatctccttcatcaccggtgaggcctccgacaagtgccagcgcgagaagcgcaagaccatcaacggcgacgacctcctctgggccatgaccacgctcggcttcgaggactacgtcgagccgctcaagctctacctccacaagttccgcgagctcgagggcgacaaggcggcc

SEQ ID NO: 80 - protein, 118 aa, Zea mays
MPDSDNDESGGPSNADFSSPREQDRFLPIANVSRIMKKALPANAKISKDGKETVQECVSEFISFITGEASDKCQREKRKTINGDDLLWAMTTLGFEDYVEPLKLYLHKFRELEGDKAA

SEQ ID NO: 81 - DNA, 336 bp, Zea mays
  CDS (1)..(336): NF-YB (AC192373)
atgtcggaggtggaggccaacgccggcggcgggggcaaggagcaggaccgattcctccctgtcgccaacatcgggcgcatcatgcgccgcgcggtgctggagaacggaaagatcgctagggacgcgagggagtccatccaggagtgcgtctccgagttcatggcgagtgacaagtgcgtcaaggagaggcgaaagaccatcaacgacgacgacatcatctggtccttgggtacgctcggcttcgaggaatacgttgagcccctcaagatctacctgaacaattaccgggagcagtatgggtacttgcttagcatagcagttcaatatggagtttga

SEQ ID NO: 82 - protein, 111 aa, Zea mays
MSEVEANAGGGGKEQDRFLPVANIGRIMRRAVLENGKIARDARESIQECVSEFMASDKCVKERRKTINDDDIIWSLGTLGFEEYVEPLKIYLNNYREQYGYLLSIAVQYGV

SEQ ID NO: 83 - DNA, 336 bp, Zea mays
  CDS (1)..(336): NF-YB (AC204642)
atgtcggaggtggaggccaacgccggcggcgggggcaaggagcaggaccgattcctccctgtcgccaacatcgggcgcatcatgcgccgcgcggtgctggagaacggaaagatcgctagggacgcgagggagtccatccaggagtgcgtctccgagttcgcgagtgacaagtgcgtcaaggagaggcgaaagaccatcaacgacgacgacatcatctggtccttgggtacgctcggcttcgaggaatacgttgagcccctcaagatctacctgaacaattaccgggagcagcagtatgggtacttgcttagcatagcagttcaatatggagtttga

SEQ ID NO: 84 - protein, 111 aa, Zea mays
MSEVEANAGGGGKEQDRFLPVANIGRIMRRAVLENGKIARDARESIQECVSEFASDKCVKERRKTINDDDIIWSLGTLGFEEYVEPLKIYLNNYREQQYGYLLSIAVQYGV

SEQ ID NO: 85 - DNA, 594 bp, Zea mays
  CDS (1)..(594): NF-YB (AC205067)
atgagcaaagctcaaggaagtaatgatcatcaacatgaacatgaagatcccgaaggttcaaaaccactagaagagtacaccatcccgaaaggcaccataacgcgtatcatgcgccaagtcctgccgcaagactcgagggtcaccggcggcgccaaggagaccatggaccagtgcatcgtccaattctccacggccctcgtacgcgccgccacgcaagagtgccggcgagaccgccgcctcaccatcaccgccgacgacctgatcgtcggctttgctaacctcggactggctgactacgtgcagcccatgtccgtgtacctacgtctgtaccgtgagaccgttaataaccagcagcaggccgtagcaccgccgtcaccgaccgtgcagagagggacgacgacggcagtgccgccgccgccgccgaacctcacgctgcaactgggcctgccgtccgtgcctgatgtcaccgagctggcgcgtgatactgacgtgtacgcgctgtggcatggagctgcaccggcagcggggagcacgagcgcctcctcggtggcgccgatgccgccacctgctgctgatggtgatgaagatgagtga

SEQ ID NO: 86 - protein, 197 aa, Zea mays
MSKAQGSNDHQHEHEDPEGSKPLEEYTIPKGTITRIMRQVLPQDSRVTGGAKETMDQCIVQFSTALVRAATQECRRDRRLTITADDLIVGFANLGLADYVQPMSVYLRLYRETVNNQQQAVAPPSPTVQRGTTTAVPPPPPNLTLQLGLPSVPDVTELARDTDVYALWHGAAPAAGSTSASSVAPMPPPAADGDEDE

SEQ ID NO: 87 - DNA, 834 bp, Zea mays
  CDS (1)..(834): NF-YB (AC210260-FG026)
atggactccagcagcttcctccctgccgccggcgcggagaatggctcggcggcgggcggcgccaacaatggcggcgctgctcagcagcatgcggcgccggcgatccgcgagcaggaccggctgatgccgatcgcgaacgtgatccgcatcatgcggcgcgtgctgccggcgcacgccaagatctcggacgacgccaaggagacgatccaggagtgcgtgtcggagtacatcagcttcatcacgggggaggccaacgagcggtgccagcgggagcagcgcaagaccatcaccgccgaggacgtgctgtgggccatgagccgcctcggcttcgacgactacgtcgagccgctcggcgcctacctccaccgctaccgcgagttcgagggcgacgcgcgcggcgtcgggctcgtcccgggggccgccccatcgcgcggcggcgaccaccacccgcactccatgtcgccagcggcgatgctcaagtcccgcgggccagtctccggagccgccatgctaccgcaccaccaccaccaccacgacatgcagatgcacgccgccatgtacgggggaacggccgtgcccccgccggccgggcctcctcaccacggcgggttcctcatgccacacccacagggtagtagccactacctgccttacgcgtacgagcccacgtacggcggtgagcacgccatggctgcatactatggaggcgccgcgtacgcgcccggcaacggcgggagcggcgacggcagtggcagtggcggcggtggcgggagcgcgtcgcacacaccgcagggcagcggcggcttggagcacccgcacccgttcgcgtacaag

SEQ ID NO: 88 - protein, 278 aa, Zea mays
MDSSSFLPAAGAENGSAAGGANNGGAAQQHAAPAIREQDRLMPIANVIRIMRRVLPAHAKISDDAKETIQECVSEYISFITGEANERCQREQRKTITAEDVLWAMSRLGFDDYVEPLGAYLHRYREFEGDARGVGLVPGAAPSRGGDHHPHSMSPAAMLKSRGPVSGAAMLPHHHHHHDMQMHAAMYGGTAVPPPAGPPHHGGFLMPHPQGSSHYLPYAYEPTYGGEHAMAAYYGGAAYAPGNGGSGDGSGSGGGGGSASHTPQGSGGLEHPHPFAYK

SEQ ID NO: 89 - DNA, 252 bp, Zea mays
  CDS (1)..(252): NF-YB (ZmEvi043847)
atgccgatcgcgaacgtcatccgcatcatgcggcgcgtgctgccggcgcacgccaagatctcggacgacgccaaggagacgatccaggagtgcgtgtcggagtacatcagcttcatcacgggggaggccaacgagcggtgccagcgggagcagcgcaagaccatcaccgccgaggacgtgctgtgggccatgagccgcctcggcttcgacgactacgtcgagccgctcagcgtctacctccaccgctaccgc

SEQ ID NO: 90 - protein, 84 aa, Zea mays
MPIANVIRIMRRVLPAHAKISDDAKETIQECVSEYISFITGEANERCQREQRKTITAEDVLWAMSRLGFDDYVEPLSVYLHRYR

SEQ ID NO: 91 - DNA, 498 bp, Zea mays
  CDS (1)..(498): NF-YB (AC208347-FGT020)
agccatacttccaatttcactggtttttcccaattagaacatccccaaccccaaagaaattctagggcatcaagttcaaccacacatgatgccaacgttaggcatgacaacaacctcttgcccattgccaatgtcgggaggatcatgaaggacgccctccctccgcaagccaagatctcgaagcacgccaaggagaccatccaagagtgcacgacggagttcgtgggcttcgtcaccggcgaggcctccgagcggtgccgccgggagaggcggaagaccatcaacggcgacgacatctgccatgccatgaggagccttggcctcgaccactacgccgacgccatgcgcaggtacctgcagaggtatcgtgagaccgaggagctagcggcagcactcaacagtggcggtggtggccacgatggcaatgccattcagattgatgtgagggatgagttgtccatcttcaagggtagcaaccagcaaggtggcagagattaa

SEQ ID NO: 92 - protein, 165 aa, Zea mays
SHTSNFTGFSQLEHPQPQRNSRASSSTTHDANVRHDNNLLPIANVGRIMKDALPPQAKISKHAKETIQECTTEFVGFVTGEASERCRRERRKTINGDDICHAMRSLGLDHYADAMRRYLQRYRETEELAAALNSGGGGHDGNAIQIDVRDELSIFKGSNQQGGRD

SEQ ID NO: 93 - DNA, 537 bp, Zea mays
  CDS (1)..(537): NF-YB (AC208347-FGT011)
gccgatcaccaccaccaccaccaccacgggcatccgccggacgggcccggcggcgcgggggaccagctggaggtaatcaaggagcaggaccggctgctgcccatcgccaacgtcggccgcatcatgaagcagatcctgccgcccaacgccaagatctccaaggaggccaaggagacgatgcaggagtgcgtgtccgagttcatcagcttcgtcacgggcgaggcctccgacaagtgccacaaggagaagcgcaagaccgtcaacggcgacgacgtctgctgcgccttcggcgcgctcggcttcgacgactacgtcgaccccatgcgcaggtacctgcacaagtaccgcgagctcgagggcgaccgcgccgcgtcctcccgcggaggcggaggcggaccggccggcgccgcagaccccgcgtccgcctcggccgccgccggtcccagccccagcgccgcatcagccggtcatttcatgttcggcgccgccatggaccggcccgacaacaactcatccgccggcgccaggcccttttaa

SEQ ID NO: 94 - protein, 178 aa, Zea mays
ADHHHHHHHGHPPDGPGGAGDQLEVIKEQDRLLPIANVGRIMKQILPPNAKISKEAKETMQECVSEFISFVTGEASDKCHKEKRKTVNGDDVCCAFGALGFDDYVDPMRRYLHKYRELEGDRAASSRGGGGGPAGAADPASASAAAGPSPSAASAGHFMFGAAMDRPDNNSSAGARPF

SEQ ID NO: 95 - DNA, 354 bp, Zea mays; identical to SEQ ID NO: 79
  CDS (1)..(354): NF-YB (ZmEvi090686)

SEQ ID NO: 96 - protein, 118 aa, Zea mays; identical to SEQ ID NO: 80

SEQ ID NO: 97 - DNA, 432 bp, Zea mays
  CDS (1)..(432): NF-YB (AC210719)
atgccggactcggacaacgactccggcgggccgagcaacgccgggggcgagctgtcgtcgccgcgggagcaggaccggttcctgcccatcgccaacgtgagccggatcatgaagaaggcgctcccggccaacgccaagatcagcaaggacgccaaggagacggtgcaggagtgcgtgtccgagttcatctccttcatcaccggcgaggcctccgacaagtgccagcgcgagaagcgcaagaccatcaacggcgacgacctgctgtgggccatgaccacgctcggcttcgaggactacgtcgagccgctcaagcactacctgcacaagttccgcgagatcgagggcgagagggccgccgcgtccgccggcgcctcgggctcgcagcagcagcagcagcaaggcgagctgcccagaggcgccgcccatggcggc

SEQ ID NO: 98 - protein, 144 aa, Zea mays
MPDSDNDSGGPSNAGGELSSPREQDRFLPIANVSRIMKKALPANAKISKDAKETVQECVSEFISFITGEASDKCQREKRKTINGDDLLWAMTTLGFEDYVEPLKHYLHKFREIEGERAAASAGASGSQQQQQQGELPRGAAHGG

SEQ ID NO: 99 - DNA, 777 bp, Zea mays
  CDS (1)..(777): NF-YB (AC204710)
atgaagaacaggaagggctacgggcaccagggccacctgctgagccccgtgggcagcccgctgtcggacaacgagtccggcgccgcggcagcggccggcggcggcgggtgcgggagcagcgtggggtactgcggcggcggcggcggtgagtcgccggccaaggagcaagaccggttcctgccgatcgccaacgtgtcgcgcatcatgaagcgctccctgccggcgaacgccaagatctccaaggaggccaaggagacggtgcaggagtgcgtgtccgagttcatcagcttcgtcacgggggaggcctccgacaagtgccagcgcgagaagcgcaagaccatcaacggcgacgacctgctctgggccatgaccacgctcggcttcgaggcctacgtcgccccgctcaagtcctacctcaaccgctaccgcgaggccgagggcgagaaggccgccgtgctcggcggcggcgcgcgccacggcgaaggcggcggcgcggcggacgacgccggcccactcgccgcgggcggcggggccggggacggtgtcgaccgcgcgggccacgacgacgacgcgcacgtcgggctcatgatgggagccagcagcgtcgggttcggctctggcggcggggcggcgccgtcgtactacgcggcggcatcacggaaggcgtacggcgcgggggaagggtccaaggtgatggagttcgaaggcgaggaggagaacggcggcgtgcagagggggaggggatttgccagccacctccacggcgccgtgcaatgg

SEQ ID NO: 100 - protein, 259 aa, Zea mays
MKNRKGYGHQGHLLSPVGSPLSDNESGAAAAAGGGGCGSSVGYCGGGGGESPAKEQDRFLPIANVSRIMKRSLPANAKISKEAKETVQECVSEFISFVTGEASDKCQREKRKTINGDDLLWAMTTLGFEAYVAPLKSYLNRYREAEGEKAAVLGGGARHGEGGGAADDAGPLAAGGGAGDGVDRAGHDDDAHVGLMMGASSVGFGSGGGAAPSYYAAASRKAYGAGEGSKVMEFEGEEENGGVQRGRGFASHLHGAVQW

SEQ ID NO: 101 - DNA, 357 bp, Zea mays
  CDS (1)..(357): NF-YB (ZmEvi027465)
atgcgccgcgcggtgacggagaacggaaagatcgctagggatgccagggagtccatccaggagtgtgtctccgagttcatcagcttcatcaccagcgaggcgagtgataagtgcgtcaaggagaggcgaaagaccatcaacgacgacgacatcatttggtccttgggtacgctcggctttgaggaatacgttgagcccctcaagatctacctcaacaattaccaggaggtatcattccagttatcctcctctaatcctgtttattgggtatatattcgttcattatatcttttgttcaactgccaaggcacaatatgtgcgcgtttaggagacaaatttggatttatgtcattctaa

SEQ ID NO: 102 - protein, 118 aa, Zea mays
MRRAVTENGKIARDARESIQECVSEFISFITSEASDKCVKERRKTINDDDIIWSLGTLGFEEYVEPLKIYLNNYQEVSFQLSSSNPVYWVYIRSLYLLFNCQGTICARLGDKFGFMSF

SEQ ID NO: 103 - DNA, 1140 bp, Zea mays
  CDS (1)..(1140): NF-YB (AC212092)
tcggaggtggaggccaacgccggcggcgggggaaagaagcaagaccgattcctgcctgtcgccaacatcgggcgcatcatgcgccgcgcggtgccggagaacggaaagatcgctagggacgccagggagtccatccaggagtgcatctccgagttcatggcgagtgacaagtgcgtcaaggagaggcgaaagaccatcaacggtgacgacatcatctggtccttgggtacgctcggcttcgaggaatacgttgagcccctcaagatctacctcaacaaataccgggaggtcccattccagttatcctcctctaatcctatttactggggtgacacaaagggttcaaagtcttctgatcagaatggaaagaaacagatgttacccaatggtgaacttggatcatcgaggactaagtacaacaacatggagccggatgctattgatttctttggggaatgtatgaatagtccccaaaatggtcgtactccgcttgcaaatgagatatatgagcaaatggttgcagagaaggaaagagagctagaagaaggagaagcacaaaagtcaccctccaagattgttgctgatagtctcagccagatcagtcgctcatccaccttccttccaaacattggtgtctgtttgaatgcttcaaacctttgtggtggaacctttgtatgtttgaacctctgttgtggaaagtactacgtaatgtccgaagagacgggcttcgatccgataccagctgtgacggaacctcccaaggtattaggcccacctacagatgtccttgtcctaaggacctcagacagtcatgcacgtacggcagaaacagcagaactcagctgtgtcatcacctacaacaacatgggtttgaaaaccctgagtacgaagtgtatttttgcaagtcttacccgtcaaaggaaaaagactctcaaggatatgctggtttttttgggaatcaaggtttctcaaaaccagaatgagttcacgccaaccgagagcacagatactccaccgttcagcctcttgccatggagggtacacgctactctcgccatctctccactcccattgcatggtggcctttctggtattggtccgaccgaggcaaaacttacccatgatgaggcatctggctag

SEQ ID NO: 104 - protein, 379 aa, Zea mays
SEVEANAGGGGKKQDRFLPVANIGRIMRRAVPENGKIARDARESIQECISEFMASDKCVKERRKTINGDDIIWSLGTLGFEEYVEPLKIYLNKYREVPFQLSSSNPIYWGDTKGSKSSDQNGKKQMLPNGELGSSRTKYNNMEPDAIDFFGECMNSPQNGRTPLANEIYEQMVAEKERELEEGEAQKSPSKIVADSLSQISRSSTFLPNIGVCLNASNLCGGTFVCLNLCCGKYYVMSEETGFDPIPAVTEPPKVLGPPTDVLVLRTSDSHARTAETAELSCVITYNNMGLKTLSTKCIFASLTRQRKKTLKDMLVFLGIKVSQNQNEFTPTESTDTPPFSLLPWRVHATLAISPLPLHGGLSGIGPTEAKLTHDEASG

SEQ ID NO: 105 - DNA, 492 bp, Arabidopsis thaliana
  CDS (1)..(492): NF-YB (AT5G08190)
atggatccgatggatatagttggaaaatcaaaggaagatgcttcgcttccgaaagctaccatgacgaaaattataaaggagatgttacccgcagatgttcgtgttgctagagatgctcaagatctattgattgagtgttgtgtagagttcataaatcttatatcgtcggagtcgaatgaagtgtgtaacaaagaggataaacgaacaatagcccctgagcacgtactcaaagctttacaggttcttggctttggggaatacgtagaagaagtttatgccgcgtatgagcaacataaatatgaaaccatgcaggattcgcagagaagtgtgaagatgaatagcggagcagagatgacggaagaagaggcggcagcggaacaacagaggatgtttgcagaagctcgagcaagaatgaatggaggtgttacagttcctcaaccagagcaactagaagaaccccaacaacaacaacaaacaagtctacaaagctaa

SEQ ID NO: 106 - protein, 163 aa, Arabidopsis thaliana
MDPMDIVGKSKEDASLPKATMTKIIKEMLPADVRVARDAQDLLIECCVEFINLISSESNEVCNKEDKRTIAPEHVLKALQVLGFGEYVEEVYAAYEQHKYETMQDSQRSVKMNSGAEMTEEEAAAEQQRMFAEARARMNGGVTVPQPEQLEEPQQQQQTSLQS

SEQ ID NO: 107 - DNA, 480 bp, Arabidopsis thaliana
  CDS (1)..(480): NF-YB (AT5G23090)
atggatccaatggatatagtcggcaaatccaaggaagacgcttctcttccaaaagctacgatgactaaaattataaaggagatgttaccaccagatgttcgtgttgcaagagatgctcaagatcttctcattgaatgttgtgtagagtttataaatcttgtatcttcagaatctaatgatgtttgtaacaaagaggataaacggacgattgctcctgagcatgttctcaaggcattacaggttcttggttttggagaatacattgaagaagtctatgctgcgtatgagcaacataagtatgaaacaatgcaggacacacagaggagcgtgaaatggaaccctggagctcaaatgactgaggaggaagcagcagctgagcaacaacgtatgtttgcagaagcacgtgcaagaatgaatggaggtgtttcggttcctcaacctgaacatccagaaactgaccagagaagtccgcaaagctaa

SEQ ID NO: 108 - protein, 159 aa, Arabidopsis thaliana
MDPMDIVGKSKEDASLPKATMTKIIKEMLPPDVRVARDAQDLLIECCVEFINLVSSESNDVCNKEDKRTIAPEHVLKALQVLGFGEYIEEVYAAYEQHKYETMQDTQRSVKWNPGAQMTEEEAAAEQQRMFAEARARMNGGVSVPQPEHPETDQRSPQS

SEQ ID NO: 109 - protein, 275 aa, Arabidopsis thaliana
  MISC_FEATURE (1)..(275): NF-YB (gi_15225884)
MESEKVVVDELPLAIVRRVVKKKLSECSPDYDVSIHKEALLAFSESARIFIHYLSATANDFCKDARRQTMKADDVFKALEEMDFSEFLEPLKSSLEDFKKKNAGKKAGAAAASYPAGGAALKSSSGTASKPKETKKRKQEEPSTQKGARKSKIDEETKRNDEETENDNTEEENGNDEEDENGNDEEDENDDENTEENGNDEENDDENTEENGNDEENEKEDEENSMEENGNESEESGNEDHSMEENGSGVGEDNENEDGSVSGSGEEVESDEEDE

SEQ ID NO: 110 - protein, 234 aa, Arabidopsis thaliana
  MISC_FEATURE (1)..(234): NF-YB (gi_30695265)
MERGGFHGYRKLSVNNTTPSPPGLAANFLMAEGSMRPPEFNQPNKTSNGGEEECTVREQDRFMPIANVIRIMRRILPAHAKISDDSKETIQECVSEYISFITGEANERCQREQRKTITAEDVLWAMSKLGFDDYIEPLTLYLHRYRELEGERGVSCSAGSVSMTNGLVVKRPNGTMTEYGAYGPVPGIHMAQYHYRHQNGFVFSGNEPNSKMSGSSSGASGARVEVFPTQQHKY

SEQ ID NO: 111 - protein, 238 aa, Arabidopsis thaliana
  MISC_FEATURE (1)..(238): NF-YB (gi_42562232)
MERGAPFSHYQLPKSISELNLDQHSNNPTPMTSSVVVAGAGDKNNGIVVQQQPPCVAREQDQYMPIANVIRIMRKTLPSHAKISDDAKETIQECVSEYISFVTGEANERCQREQRKTITAEDILWAMSKLGFDNYVDPLTVFINRYREIETDRGSALRGEPPSLRQTYGGNGIGFHGPSHGLPPPGPYGYGMLDQSMVMGGGRYYQNGSSGQDESSVGGGSSSSINGMPAFDHYGQYK

SEQ ID NO: 112 - DNA, 420 bp, Arabidopsis thaliana
  CDS (1)..(420): NF-YB (AT1G09030)
atgacagacgaagatagattgttgccaatagccaatgtagggagacttatgaagcaaatcctaccatcaaatgcaaagatctcaaaagaagcaaaacaaacagttcaagaatgtgcaacagagttcataagctttgttacatgcgaagcatcagagaagtgccacagggagaatcggaagacggtgaatggagacgacatctggtgggctctcagcactctcggcctcgataactatgctgacgccgtgggtaggcatcttcacaagtaccgtgaagccgagagagaaagaactgagcacaacaaaggtagcaatgatagtgggaatgagaaagaaaccaacactagaagtgatgtacagaaccaatcgacaaaatttattagagttgttgagaagggaagcagctcctcggcccgttga

SEQ ID NO: 113 - protein, 139 aa, Arabidopsis thaliana
MTDEDRLLPIANVGRLMKQILPSNAKISKEAKQTVQECATEFISFVTCEASEKCHRENRKTVNGDDIWWALSTLGLDNYADAVGRHLHKYREAERERTEHNKGSNDSGNEKETNTRSDVQNQSTKFIRVVEKGSSSSAR

SEQ ID NO: 114 - protein, 160 aa, Arabidopsis thaliana
  MISC_FEATURE (1)..(160): NF-YB (gi_15227134)
MAGNYHSFQNPIPRYQNYNFGSSSSNHQHEHDGLVVVVEDQQQEESMMVKEQDRLLPIANVGRIMKNILPANAKVSKEAKETMQECVSEFISFVTGEASDKCHKEKRKTVNGDDICWAMANLGFDDYAAQLKKYLHRYRVLEGEKPNHHGKGGPKSSPDN

SEQ ID NO: 115 - DNA, 687 bp, Arabidopsis thaliana
  CDS (1)..(687): NF-YB transcription factor (AT3G53340)
atggccgaatcgcaaaccggtggtggtggtggtggaagccatgagagtggcggtgatcagagcccgaggtctttgaatgttcgtgagcaggacaggtttcttccgattgctaacataagccgtatcatgaagagaggtttacctctaaatggcaaaatcgctaaagatgctaaagagactatgcaggaatgtgtctctgaattcatcagcttcgtcaccagcgaggctagtgataagtgccaaagagagaaaaggaagaccatcaatggagatgatttgctttgggctatggccactttaggattcgaagattacatcgatcccctcaaggtttacctgatgcgatatagagagatggagggtgacactaaaggatcaggaaaaggcggggaatcgagtgcaaagagagatggtcaaccaagccaagtgtctcagttctcgcaggttcctcaacaaggctcattctcacagggtccttatggaaactctcaatctctgaggttcggcaatagcatcgagcatcttgaagtgttaatgagtagtactaggacactattcatcacaatcttccgagactcgactatgcctgttgtgtctgagaatctgagtgatccactttccatagatatggattgtgaagctatttatcaccacttcattggcctgttgattctttcatgcaagtga

SEQ ID NO: 116 - protein, 228 aa, Arabidopsis thaliana
MAESQTGGGGGGSHESGGDQSPRSLNVREQDRFLPIANISRIMKRGLPLNGKIAKDAKETMQECVSEFISFVTSEASDKCQREKRKTINGDDLLWAMATLGFEDYIDPLKVYLMRYREMEGDTKGSGKGGESSAKRDGQPSQVSQFSQVPQQGSFSQGPYGNSQSLRFGNSIEHLEVLMSSTRTLFITIFRDSTMPVVSENLSDPLSIDMDCEAIYHHFIGLLILSCK

SEQ ID NO: 117 - DNA, 519 bp, Arabidopsis thaliana
  CDS (1)..(519): NF-YB (AT2G37060)
atggcggagtcgcaggccaagagtcccggaggctgtggaagccatgagagtggtggagatcaaagtcccaggtcgttacatgttcgtgagcaagataggtttcttccgattgctaacataagccgtatcatgaaaagaggtcttcctgctaatgggaaaatcgctaaagatgctaaggagattgtgcaggaatgtgtctctgaattcatcagtttcgtcaccagcgaagcgagtgataaatgtcaaagagagaaaaggaagactattaatggagatgatttgctttgggcaatggctactttaggatttgaagactacatggaacctctcaaggtttacctgatgagatatagagagatggagggtgacacaaagggatcagcaaaaggtggggatccaaatgcaaagaaagatgggcaatcaagccaaaatggccagttctcgcagcttgctcaccaaggtccttatgggaactctcaagctcagcagcatatgatggttccaatgccgggaacagac

SEQ ID NO: 118 - protein, 173 aa, Arabidopsis thaliana
MAESQAKSPGGCGSHESGGDQSPRSLHVREQDRFLPIANISRIMKRGLPANGKIAKDAKEIVQECVSEFISFVTSEASDKCQREKRKTINGDDLLWAMATLGFEDYMEPLKVYLMRYREMEGDTKGSAKGGDPNAKKDGQSSQNGQFSQLAHQGPYGNSQAQQHMMVPMPGTD

SEQ ID NO: 119 - protein, 141 aa, Arabidopsis thaliana
  MISC_FEATURE (1)..(141): NF-YB (gi_18404885)
MADTPSSPAGDGGESGGSVREQDRYLPIANISRIMKKALPPNGKIGKDAKDTVQECVSEFISFITSEASDKCQKEKRKTVNGDDLLWAMATLGFEDYLEPLKIYLARYRELEGDNKGSGKSGDGSNRDAGGGVSGEEMPSW

SEQ ID NO: 120 - DNA, 486 bp, Arabidopsis thaliana
  CDS (1)..(486): NF-YB (AT4G14540)
atggcggattcggacaacgattcaggaggacacaaagacggtggaaatgcttcgacacgtgagcaagataggtttctaccgatcgctaacgttagcaggatcatgaagaaagcacttcctgcgaacgcaaaaatctctaaggatgctaaagaaacggttcaagagtgtgtatcggaattcataagtttcatcaccggtgaggcttctgacaagtgtcagagagagaagaggaagacaatcaacggtgacgatcttctttgggcgatgactacgctagggtttgaggactacgtggagcctctcaaggtttatctgcaaaagtatagggaggtggaaggagagaagactactacggcagggagacaaggcgataaggaaggtggaggaggaggcggtggagctggaagtggaagtggaggagctccgatgtacggtggtggcatggtgactacgatgggacatcaattttcccatcatttttcttaa

SEQ ID NO: 121 - protein, 161 aa, Arabidopsis thaliana
MADSDNDSGGHKDGGNASTREQDRFLPIANVSRIMKKALPANAKISKDAKETVQECVSEFISFITGEASDKCQREKRKTINGDDLLWAMTTLGFEDYVEPLKVYLQKYREVEGEKTTTAGRQGDKEGGGGGGGAGSGSGGAPMYGGGMVTTMGHQFSHHFS

SEQ ID NO: 122 - DNA, 648 bp, Arabidopsis thaliana
  CDS (1)..(648): NF-YB (AT2G13570)
atgactgaggagagcccagaagaagatcatgggtctcctggagtagctgaaacaaatccaggaagcccttcttcaaagaccaacaacaacaacaacaacaacaaagaacaagaccggtttcttcccattgcgaatgtcggaaggatcatgaaaaaagttcttcccggtaacggtaagatctcaaaagacgctaaagaaaccgttcaagaatgtgtctcggagttcattagtttcgtcactggtgaagcttctgacaagtgtcaaagagaaaagaggaagaccatcaatggagatgatatcatttgggctatcacaactctcggtttcgaagactacgtggctccattaaaggtctacctctgcaaatatagagacaccgaaggagagaaagttaacagcccaaaacaacaacaacaaagacaacaacaacagcagattcaacaacagaatcatcataattatcagtttcaagaacaagaccaaaacaataacaacatgtcatgtactagttacatctctcatcatcatccttctccattcctaccagtggatcatcaaccttttcccaatattgctttctctcctaaatcattgcagaaacagttcccgcagcagcatgataataacattgattcaattcactggtga

SEQ ID NO: 123 - protein, 215 aa, Arabidopsis thaliana
MTEESPEEDHGSPGVAETNPGSPSSKTNNNNNNNKEQDRFLPIANVGRIMKKVLPGNGKISKDAKETVQECVSEFISFVTGEASDKCQREKRKTINGDDIIWAITTLGFEDYVAPLKVYLCKYRDTEGEKVNSPKQQQQRQQQQQIQQQNHHNYQFQEQDQNNNNMSCTSYISHHHPSPFLPVDHQPFPNIAFSPKSLQKQFPQQHDNNIDSIHW

SEQ ID NO: 124 - DNA, 1421 bp, Sugarcane bacilliform virus
  promoter (1)..(1421): Sugarcane bacilliform virus promoter
gaagttgaagacaaagaaggtcttaaatcctggctagcaacactgaactatgccagaaaccacatcaaagatatgggcaagcttcttggcccattatatccaaagacctcagagaaaggtgagcgaaggctcaattcagaagattggaagctgatcaataggatcaagacaatggtgagaacgcttccaaatctcactattccaccagaagatgcatacattatcattgaaacagatgcatgtgcaactggatggggagcagtatgcaagtggaagaaaaacaaggcagacccaagaaatacagagcaaatctgtaggtatgccagtggaaaatttgataagccaaaaggaacctgtgatgcagaaatctatggggttatgaatggcttagaaaagatgagattgttctacttggacaaaagagagatcacagtcagaactgacagtagtgcaatcgaaaggttctacaacaagagtgctgaacacaagccttctgagatcagatggatcaggttcatggactacatcactggtgcaggaccagagatagtcattgaacacataaaagggaagagcaatggtttagctgacatcttgtccaggctcaaagccaaattagctcagaatgaaccaacggaagagatgatcctgcttacacaagccataagggaagtaattccttatccagatcatccatacactgagcaactcagagaatggggaaacaaaattctggatccattccccacattcaagaaggacatgttcgaaagaacagagcaagcttttatgctaacagaggaaccagttctactctgtgcatgcaggaagcctgcaattcagttagtgtccagaacatctgccaacccaggaaggaaattcttcaagtgcgcaatgaacaaatgccattgctggtactgggcagatctcattgaagaacacattcaagacagaattgatgaatttctcaagaatcttgaagttctgaagaccggtggcgtgcaaacaatggaggaggaacttatgaaggaagtcaccaagctgaagatagaagagcaggagttcgaggaataccaggccacaccaagggctatgtcgccagtagccgcagaagatgtgctagatctccaagacgtaagcaatgacgattgaggaggcattgacgtcagggatgaccgcagcggagagtactgggcccattcagtggatgctccactgagttgtattattgtgtgcttttcggacaagtgtgctgtccactttcttttggcacctgtgccactttattccttgtctgccacgatgcctttgcttagcttgtaagcaaggatcgcagtgcgtgtgtgacaccaccccccttccgacgctctgcctatataaggcaccgtctgtaagctcttacgatcatcggtagttcacca

(19) United States
(12) Patent Application Publication
(10) Pub. No.: US 2012/0042418 A1
(43) Pub. Date: Feb. 16, 2012

(54) Engineering NF-YB Transcription Factors for Enhanced Drought Resistance and Increased Yield in Transgenic Plants

(75) Inventor: Resham Kulkarni, Cary, NC (US)

(73) Assignee: BASF Plant Science Company GmbH, Ludwigshafen (DE)

(21) Appl. No.: 13/143,732

(22) PCT Filed: Jan. 25, 2010
(86) PCT No.: PCT/EP2010/050757

§ 371 Date: Jul. 8, 2011

Related U.S. Application Data
(60) Provisional application No. 61/147,777, filed on Jan. 28, 2009.

(51) Int. Cl. A01H 5/00; C12N 15/82; C12N 15/29; A01H 5/10

(52) U.S. Cl. 800/290; 800/298; 800/320.1; 800/320.3; 800/320; 800/320.2; 800/312; 800/314; 800/306; 800/317.1; 800/322; 800/317.3; 800/317; 800/317.4; 800/317.2; 536/23.6

(57) ABSTRACT

Polynucleotides are disclosed which are capable of enhancing yield of a plant transformed to contain such polynucleotides. Also provided are methods of using such polynucleotides and transgenic plants and agricultural products, including seeds, containing such polynucleotides as transgenes.

This application claims priority benefit of U.S. provisional patent application Ser. No. 61/147,777, filed Jan. 28, 2009, the entire contents of which are incorporated herein by reference.

### FIELD OF THE INVENTION

This invention relates generally to transgenic plants which overexpress isolated polynucleotides that encode polypeptides active in regulation of transcription, thereby improving the yield of said plants.

### BACKGROUND OF THE INVENTION

Population increases and climate change have brought the possibility of global food, feed, and fuel shortages into sharp focus in recent years. Agriculture consumes 70% of water used by people, at a time when rainfall in many parts of the world is declining. In addition, as land use shifts from farms to cities and suburbs, fewer hectares of arable land are available to grow agricultural crops. Agricultural biotechnology has attempted to meet humanity's growing needs through genetic modifications of plants that could increase crop yield, for example, by conferring better tolerance to abiotic stress responses or by increasing biomass.

Crop yield is defined herein as the number of bushels of relevant agricultural product (such as grain, forage, or seed) harvested per acre. Crop yield is impacted by abiotic stresses, such as drought, heat, salinity, and cold stress, and by the size (biomass) of the plant. Traditional plant breeding strategies are relatively slow and have in general not been successful in conferring increased tolerance to abiotic stresses. Grain yield improvements by conventional breeding have nearly reached a plateau in maize. The harvest index, i.e., the ratio of yield biomass to the total cumulative biomass at harvest, in maize has remained essentially unchanged during selective breeding for grain yield over the last hundred years. Accordingly, recent yield improvements that have occurred in maize are the result of the increased total biomass production per unit land area. This increased total biomass has been achieved by increasing planting density, which has led to adaptive phenotypic alterations, such as a reduction in leaf angle, which may reduce shading of lower leaves, and tassel size, which may increase harvest index.

When soil water is depleted or if water is not available during periods of drought, crop yields are restricted. Plant water deficit develops if transpiration from leaves exceeds the supply of water from the roots. The available water supply is related to the amount of water held in the soil and the ability of the plant to reach that water with its root system. Transpiration of water from leaves is linked to the fixation of carbon dioxide by photosynthesis through the stomata. The two processes are positively correlated so that high carbon dioxide influx through photosynthesis is closely linked to water loss by transpiration. As water transpires from the leaf, leaf water potential is reduced and the stomata tend to close in a hydraulic process limiting the amount of photosynthesis. Since crop yield is dependent on the fixation of carbon dioxide in photosynthesis, water uptake and transpiration are contributing factors to crop yield. Plants which are able to use less water to fix the same amount of carbon dioxide or which are able to function normally at a lower water potential have the potential to conduct more photosynthesis and thereby to produce more biomass and economic yield in many agricultural systems.

Agricultural biotechnologists have used assays in model plant systems, greenhouse studies of crop plants, and field trials in their efforts to develop transgenic plants that exhibit increased yield, either through increases in abiotic stress tolerance or through increased biomass. For example, water use efficiency (WUE) is a parameter often correlated with drought tolerance. Studies of a plant's response to desiccation, osmotic shock, and temperature extremes are also employed to determine the plant's tolerance or resistance to abiotic stresses.

An increase in biomass at low water availability may be due to relatively improved efficiency of growth or reduced water consumption. In selecting traits for improving crops, a decrease in water use, without a change in growth would have particular merit in an irrigated agricultural system where the water input costs were high. An increase in growth without a corresponding jump in water use would have applicability to all agricultural systems. In many agricultural systems where water supply is not limiting, an increase in growth, even if it came at the expense of an increase in water use also increases yield.

Agricultural biotechnologists also use measurements of other parameters that indicate the potential impact of a transgene on crop yield. For forage crops like alfalfa, silage corn, and hay, the plant biomass correlates with the total yield. For grain crops, however, other parameters have been used to estimate yield, such as plant size, as measured by total plant dry weight, above-ground dry weight, above-ground fresh weight, leaf area, stem volume, plant height, rosette diameter, leaf length, root length, root mass, tiller number, and leaf number. Plant size at an early developmental stage will typically correlate with plant size later in development. A larger plant with a greater leaf area can typically absorb more light and carbon dioxide than a smaller plant and therefore will likely gain a greater weight during the same period. There is a strong genetic component to plant size and growth rate, and so for a range of diverse genotypes plant size under one environmental condition is likely to correlate with size under another. In this way a standard environment is used to approximate the diverse and dynamic environments encountered at different locations and times by crops in the field.

Harvest index is relatively stable under many environmental conditions, and so a robust correlation between plant size and grain yield is possible. Plant size and grain yield are intrinsically linked, because the majority of grain biomass is dependent on current or stored photosynthetic productivity by the leaves and stem of the plant. As with abiotic stress tolerance, measurements of plant size in early development, under standardized conditions in a growth chamber or greenhouse, are standard practices to measure potential yield advantages conferred by the presence of a transgene.

NF-Y transcription factors bind to the CCAAT-box consensus to regulate a diverse set of genes as a trimer of three proteins comprising NF-YA, NF-YB and NF-YC that binds to DNA with high affinity and specificity to activate transcription. The NF-Y subunits NF-YA, NF-YB, NF-YC are also referred to as CBF-B/HAP2, CBF-A/HAP3 and CBF-C/HAP5 respectively. All three subunits contain conserved core domains that comprise the histone fold, which is a core of three helices, where the long middle helix is flanked at each end by the shorter one. These domains are involved in different functions including DNA-binding, subunit interactions, and nuclear transport.

Single genes for the NF-Y subunits are found in fungi and animals, however, multiple genes are present in plants. In Arabidopsis thaliana there are multiple genes that encode the different NF-Y subunits. NF-YA has 10 genes; NF-YB, 13 genes; NF-YC, 13 genes. In the rice genome, a similar diversity exists. For NF-YA, there are 10 genes; NF-YB, 11 genes; NF-YC, 7 genes. Similarly, in the Triticum aestivum genome, the NF-YA gene family consists of 10 genes, the NF-YB gene family consists of 11 genes, and 14 genes comprise the NF-YC gene family. In all cases, the different NF-Y genes in each species are expressed differently among tissues and environmental treatments, indicating that each is under independent transcriptional regulation.

U.S. Patent Application Publication 2005/0022266 discloses that transformation of a Zea mays Hap3 transcription factor into plants under control of the CaMV 35S promoter improves drought tolerance. U.S. Patent Application Publication 2008/040973 discloses that use of the 35S promoter with this transcription factor results in reduced yield when plants are grown under water sufficient conditions. US 2008/040973 further discloses that use of an enhancerless rice actin promoter with the Z. mays NF-YB gene causes transgenic plants to produce less NF-YB protein, and that such transgenic plants show enhanced yield under both drought and water sufficient conditions.

U.S. Pat. No. 7,482,511 discloses the Physcomitrella patens NF-YB transcription factor EST265 as PpCABF-3, and U.S. Pat. No. 7,164,057 discloses the P. patens NF-YB transcription factor EST69 as PpCABF-1.

U.S. Patent Application Publication 2007/0199107 discloses that transgenic plants in which certain NF-YB transcription factors are overexpressed as transgenes demonstrate early flowering, as compared to wild type plants which do not comprise the transgene.

While attenuating the level of NF-YB protein in transgenic plants has improved yield, a need continues to exist to increase yield of crop plants further.

### SUMMARY OF THE INVENTION

The present invention provides novel functional combinations of the NF-Y complex by combining different domains from either different species or different members of the gene family within a species. The NF-YB polynucleotides and chimeric polynucleotides and polypeptides set forth in Table 1 are capable of improving yield of transgenic plants.

TABLE 1

| | | Polynucleotide | Amino acid |
|---|---|---|---|
| Gene Name | Organism | SEQ ID NO | SEQ ID NO |
| EST265 | P patens | 1 | 2 |
| AT5G47640 | A. thaliana | 3 | 4 |
| NM_001112582.1 | Z. mays | 5 | 6 |
| NFYB-C1 | Artificial | 7 | 8 |
| NFYB-C2 | Artificial | 9 | 10 |
| NFYB-C3 | Artificial | 11 | 12 |
| NFYB-C4 | Artificial | 13 | 14 |
| NFYB-C5 | Artificial | 15 | 16 |
| NFYB-C6 | Artificial | 17 | 18 |
| NFYB-C7 | Artificial | 19 | 20 |
| NFYB-C8 | Artificial | 21 | 22 |
| NFYB-C9 | Artificial | 23 | 24 |
| NFYB-C10 | Artificial | 25 | 26 |
| NFYB-C11 | Artificial | 27 | 28 |
| NFYB-C12 | Artificial | 29 | 30 |
| EST69 | P. patens | 31 | 32 |
| ZM58019377 | Z. mays | 33 | 34 |
| ZM61020893 | Z. mays | 35 | 36 |
| ZM62014459 | Z. mays | 37 | 38 |
| ZM62260706 | Z. mays | 39 | 40 |
| ZM59456239 | Z. mays | 41 | 42 |
| ZM59473285 | Z. mays | 43 | 44 |
| ZM59153552 | Z. mays | 45 | 46 |
| ZM62055702 | Z. mays | 47 | 48 |
| ZM67286704 | Z. mays | 49 | 50 |
| ZM65405678 | Z. mays | 51 | 52 |
| ZM62083966 | Z. mays | 53 | 54 |
| ZmEvi061009.1 | Z. mays | 55 | 56 |
| ZmEvi061009.2 | Z. mays | — | 57 |
| ZmEvi061009.3 | Z. mays | — | 58 |
| AC198485 | Z. mays | 59 | 60 |
| AC203785 | Z. mays | 61 | 62 |
| AC210260 | Z. mays | 63 | 64 |
| AC205600 | Z. mays | 65 | 66 |
| ZmEvi005132 | Z. mays | 67 | 68 |
| AC203676 | Z. mays | 69 | 70 |
| AC188837 | Z. mays | 71 | 72 |
| AC203033 | Z. mays | 73 | 74 |
| AC187072 | Z. mays | 75 | 76 |
| ZmEvi013724 | Z. mays | 77 | 78 |
| AC204839 | Z. mays | 79 | 80 |
| AC192373 | Z. mays | 81 | 82 |
| AC204642 | Z. mays | 83 | 84 |
| AC205067 | Z. mays | 85 | 86 |
| AC210260-FG026 | Z. mays | 87 | 88 |
| ZmEvi043847 | Z. mays | 89 | 90 |
| AC208347-FGT020 | Z. mays | 91 | 92 |
| AC208347-FGT011 | Z. mays | 93 | 94 |
| ZmEvi090686 | Z. mays | 95 | 96 |
| AC210719 | Z. mays | 97 | 98 |
| AC204710 | Z. mays | 99 | 100 |
| ZmEvi027465 | Z. mays | 101 | 102 |
| AC212092 | Z. mays | 103 | 104 |
| AT5G08190 | A. thaliana | 105 | 106 |
| AT5G23090 | A. thaliana | 107 | 108 |
| gi_15225884 | A. thaliana | — | 109 |
| gi_30695265 | A. thaliana | — | 110 |
| gi_42562232 | A. thaliana | — | 111 |
| AT1G09030 | A. thaliana | 112 | 113 |
| gi_15227134 | A. thaliana | — | 114 |
| AT3G53340 | A. thaliana | 115 | 116 |
| AT2G37060 | A. thaliana | 117 | 118 |
| gi_18404885 | A. thaliana | — | 119 |
| AT4G14540 | A. thaliana | 120 | 121 |
| AT2G13570 | A. thaliana | 122 | 123 |

In one embodiment, the invention provides a transgenic plant transformed with an expression cassette comprising, in operative association, an isolated polynucleotide encoding a promoter; and an isolated polynucleotide encoding a full-length polypeptide which is a chimeric NF-YB transcription factor; wherein the transgenic plant demonstrates increased yield as compared to a wild type plant of the same variety which does not comprise the expression cassette.

In a further embodiment, the invention provides a seed produced by the transgenic plant of the invention, wherein the seed is true breeding for a transgene comprising the expression vectors described above. Plants derived from the seed of the invention demonstrate increased tolerance to an environmental stress, and/or increased plant growth, and/or increased yield, under normal or stress conditions as compared to a wild type variety of the plant.

In a still another aspect, the invention concerns products produced by or from the transgenic plants of the invention, their plant parts, or their seeds, such as a foodstuff, feedstuff, food supplement, feed supplement, fiber, cosmetic or pharmaceutical.

The invention further provides certain isolated polynucleotides identified in Table 1, and certain isolated polypeptides identified in Table 1. The invention is also embodied in recombinant vector comprising an isolated polynucleotide of the invention.

In yet another embodiment, the invention concerns a method of producing the aforesaid transgenic plant, wherein the method comprises transforming a plant cell with an expression vector comprising an isolated polynucleotide of the invention, and generating from the plant cell a transgenic plant that expresses the polypeptide encoded by the polynucleotide. Expression of the polypeptide in the plant results in increased tolerance to an environmental stress, and/or growth, and/or yield under normal and/or stress conditions as compared to a wild type variety of the plant.

In still another embodiment, the invention provides a method of increasing a plant's tolerance to an environmental stress, and/or growth, and/or yield. The method comprises the steps of transforming a plant cell with an expression cassette comprising an isolated polynucleotide of the invention, and generating a transgenic plant from the plant cell, wherein the transgenic plant comprises the polynucleotide.

### BRIEF DESCRIPTION OF THE DRAWINGS

FIG. 1 shows the distinct domains in the NF-YB genes and their putative functions.

FIG. 2 shows a phylogenetic tree illustrating the relatedness between representatives of selected NF-YB genes. Only a representative from each group indicated in Table 2 is included in the tree. The tree was generated using the Maximum likelihood method. The genes representing groups that were selected for chimeric constructs are highlighted by the solid gray oval. The dotted ovals indicate genes representing groups that contain genes shown to confer drought tolerance.

FIG. 3 shows the P. patens, A. thaliana, and maize sequences, that were used for chimeric constructs, designated EST265 (SEQ ID NO:2), AT5G47640 (SEQ ID NO:4), NM_{—}001112582.1 (SEQ ID NO:6), including the distinct domains within the NFYB protein. The alignment was generated using Align X of Vector NTI.

FIGS. 4A-4T show an alignment of the translated sequences of selected P. patens, A. thaliana, and maize NF-YB genes designated ZM58019377 (SEQ ID NO:34), ZM61020893 (SEQ ID NO:36), ZM62014459 (SEQ ID NO:38), ZM62260706 (SEQ ID NO:40), ZM59456239 (SEQ ID NO:42), ZM59473285 (SEQ ID NO:44), ZM59153552 (SEQ ID NO:46), ZM62055702 (SEQ ID NO:48), ZM67286704 (SEQ ID NO:50), ZM65405678 (SEQ ID NO:52), ZM62083966 (SEQ ID NO:54), ZmEvi061009.1 (SEQ ID NO:56), AC198485 (SEQ ID NO:60), AC203785 (SEQ ID NO:62), AC210260 (SEQ ID NO:64), AC205600 (SEQ ID NO:66), ZmEvi005132 (SEQ ID NO:68), AC203676 (SEQ ID NO:70), AC188837 (SEQ ID NO:72), AC203033 (SEQ ID NO:74), AC187072 (SEQ ID NO:76), ZmEvi013724 (SEQ ID NO:78), AC204839 (SEQ ID NO:80), AC192373 (SEQ ID NO:82), AC204642 (SEQ ID NO:84), AC205067 (SEQ ID NO:86), AC210260-FG026 (SEQ ID NO:88), ZmEviO43847 (SEQ ID NO:90), AC208347-FGT020 (SEQ ID NO:92), AC208347-FGT011 (SEQ ID NO:94), ZmEvi090686 (SEQ ID NO:96), AC210719 (SEQ ID NO:98), AC204710 (SEQ ID NO:100), ZmEvi027465 (SEQ ID NO:102), AC212092 (SEQ ID NO:104), AT5G08190 (SEQ ID NO:106), AT5G23090 (SEQ ID NO:108), gi_{—}15225884 (SEQ ID NO:109), gi_{—}30695265 (SEQ ID NO:110), gi_{—}42562232 (SEQ ID NO:111), AT1G09030 (SEQ ID NO:113), gi_{—}15227134 (SEQ ID NO:114), AT3G53340 (SEQ ID NO:116), AT2G37060 (SEQ ID NO:118), gi_{—}18404885 (SEQ ID NO:119), AT4G14540 (SEQ ID NO:121), AT2G13570 (SEQ ID NO:123). The alignment was generated using Align X of Vector NTI.

### DETAILED DESCRIPTION OF THE PREFERRED EMBODIMENTS

Throughout this application, various publications are referenced. The disclosures of all of these publications and those references cited within those publications in their entireties are hereby incorporated by reference into this application in order to more fully describe the state of the art to which this invention pertains. The terminology used herein is for the purpose of describing specific embodiments only and is not intended to be limiting. As used herein, “a” or “an” can mean one or more, depending upon the context in which it is used. Thus, for example, reference to “a cell” can mean that at least one cell can be used.

In one embodiment, the invention provides a transgenic plant that overexpresses an isolated polynucleotide identified in Table 1 in the subcellular compartment and tissue indicated herein. The transgenic plant of the invention demonstrates an improved yield as compared to a wild type variety of the plant. As used herein, the term “improved yield” means any improvement in the yield of any measured plant product, such as grain, fruit or fiber. In accordance with the invention, changes in different phenotypic traits may improve yield. For example, and without limitation, parameters such as floral organ development, root initiation, root biomass, seed number, seed weight, harvest index, tolerance to abiotic environmental stress, leaf formation, phototropism, apical dominance, and fruit development, are suitable measurements of improved yield. Any increase in yield is an improved yield in accordance with the invention. For example, the improvement in yield can comprise a 0.1%, 0.5%, 1%, 3%, 5%, 10%, 15%, 20%, 30%, 40%, 50%, 60%, 70%, 80%, 90% or greater increase in any measured parameter. For example, an increase in the bu/acre yield of soybeans or corn derived from a crop comprising plants which are transgenic for the nucleotides and polypeptides of Table 1, as compared with the bu/acre yield from untreated soybeans or corn cultivated under the same conditions, is an improved yield in accordance with the invention.

As defined herein, a “transgenic plant” is a plant that has been altered using recombinant DNA technology to contain an isolated nucleic acid which would otherwise not be present in the plant. As used herein, the term “plant” includes a whole plant, plant cells, and plant parts. Plant parts include, but are not limited to, stems, roots, ovules, stamens, leaves, embryos, meristematic regions, callus tissue, gametophytes, sporophytes, pollen, microspores, and the like. The transgenic plant of the invention may be male sterile or male fertile, and may further include transgenes other than those that comprise the isolated polynucleotides described herein.

As used herein, the term “variety” refers to a group of plants within a species that share constant characteristics that separate them from the typical form and from other possible varieties within that species. While possessing at least one distinctive trait, a variety is also characterized by some variation between individuals within the variety, based primarily on the Mendelian segregation of traits among the progeny of succeeding generations. A variety is considered “true breeding” for a particular trait if it is genetically homozygous for that trait to the extent that, when the true-breeding variety is self-pollinated, a significant amount of independent segregation of the trait among the progeny is not observed. In the present invention, the trait arises from the transgenic expression of one or more isolated polynucleotides introduced into a plant variety. As also used herein, the term “wild type variety” refers to a group of plants that are analyzed for comparative purposes as a control plant, wherein the wild type variety plant is identical to the transgenic plant (plant transformed with an isolated polynucleotide in accordance with the invention) with the exception that the wild type variety plant has not been transformed with an isolated polynucleotide of the invention. The term “wild type” as used herein refers to a plant cell, seed, plant component, plant tissue, plant organ, or whole plant that has not been genetically modified with an isolated polynucleotide in accordance with the invention.

The term “control plant” as used herein refers to a plant cell, an explant, seed, plant component, plant tissue, plant organ, or whole plant used to compare against transgenic or genetically modified plant for the purpose of identifying an enhanced phenotype or a desirable trait in the transgenic or genetically modified plant. A “control plant” may in some cases be a transgenic plant line that comprises an empty vector or marker gene, but does not contain the recombinant polynucleotide of interest that is present in the transgenic or genetically modified plant being evaluated. A control plant may be a plant of the same line or variety as the transgenic or genetically modified plant being tested, or it may be another line or variety, such as a plant known to have a specific phenotype, characteristic, or known genotype. A suitable control plant would include a genetically unaltered or non-transgenic plant of the parental line used to generate a transgenic plant herein.

As defined herein, the term “nucleic acid” and “polynucleotide” are interchangeable and refer to RNA or DNA that is linear or branched, single or double stranded, or a hybrid thereof. The term also encompasses RNA/DNA hybrids. An “isolated” nucleic acid molecule is one that is substantially separated from other nucleic acid molecules which are present in the natural source of the nucleic acid (i.e., sequences encoding other polypeptides). For example, a cloned nucleic acid is considered isolated. A nucleic acid is also considered isolated if it has been altered by human intervention, or placed in a locus or location that is not its natural site, or if it is introduced into a cell by transformation. Moreover, an isolated nucleic acid molecule, such as a cDNA molecule, can be free from some of the other cellular material with which it is naturally associated, or culture medium when produced by recombinant techniques, or chemical precursors or other chemicals when chemically synthesized. While it may optionally encompass untranslated sequence located at both the 3′ and 5′ ends of the coding region of a gene, it may be preferable to remove the sequences which naturally flank the coding region in its naturally occurring replicon.

As used herein, the term “environmental stress” refers to a sub-optimal condition associated with salinity, drought, nitrogen, temperature, metal, chemical, pathogenic, or oxidative stresses, or any combination thereof. As used herein, the term “drought” refers to an environmental condition where the amount of water available to support plant growth or development is less than optimal. As used herein, the term “fresh weight” refers to everything in the plant including water. As used herein, the term “dry weight” refers to everything in the plant other than water, and includes, for example, carbohydrates, proteins, oils, and mineral nutrients.

Any plant species may be transformed to create a transgenic plant in accordance with the invention. The transgenic plant of the invention may be a dicotyledonous plant or a monocotyledonous plant. For example and without limitation, transgenic plants of the invention may be derived from any of the following dicotyledonous plant families: Leguminosae, including plants such as pea, alfalfa and soybean; Umbelliferae, including plants such as carrot and celery; Solanaceae, including the plants such as tomato, potato, aubergine, tobacco, and pepper; Cruciferae, particularly the genus Brassica, which includes plant such as oilseed rape, beet, cabbage, cauliflower and broccoli); and A. thaliana; Compositae, which includes plants such as lettuce; Malvaceae, which includes cotton; Fabaceae, which includes plants such as peanut, and the like. Transgenic plants of the invention may be derived from monocotyledonous plants, such as, for example, wheat, barley, sorghum, millet, rye, triticale, maize, rice, oats and sugarcane. Transgenic plants of the invention are also embodied as trees such as apple, pear, quince, plum, cherry, peach, nectarine, apricot, papaya, mango, and other woody species including coniferous and deciduous trees such as poplar, pine, sequoia, cedar, oak, and the like. Especially preferred are A. thaliana, Nicotiana tabacum, rice, oilseed rape, canola, soybean, corn (maize), cotton, and wheat.

In one embodiment, the invention provides a transgenic plant transformed with an expression cassette comprising, in operative association, an isolated polynucleotide encoding a promoter; and polynucleotide encoding a chimeric NF-YB transcription factor polypeptide wherein one or more of the N-terminal domain, the conserved central domain or the C-terminal domain differ in origin from one or more of the other domains, wherein the transgenic plant demonstrates increased yield as compared to a wild type plant of the same variety which does not comprise the expression cassette. The chimeric NF-YB transcription factor of the invention comprises, in order, an N-terminal domain, a central conserved domain, and a C-terminal domain. The N-terminal domain may be derived from P. patens, from a dicotyledonous plant, or a monocotyledonous plant. The central conserved domain may be derived from P. patens, from a dicotyledonous plant, or a monocotyledonous plant. The C-terminal domain may be derived from P. patens, from a dicotyledonous plant, or a monocotyledonous plant.

The transgenic plant of this embodiment may comprise any polynucleotide encoding a chimeric NF-YB transcription factor polypeptide. In specific embodiments, the transgenic plant of this embodiment comprises a polynucleotide encoding a full-length chimeric NF-YB transcription factor, wherein the polypeptide comprises three domains, wherein the N-terminal domain is selected from the group consisting of amino acids 1 to 31 of SEQ ID NO:2; amino acids 1 to 23 of SEQ ID NO:4; amino acids 1 to 27 of SEQ ID NO:6; amino acids 1 to 31 of SEQ ID NO:8; amino acids 1 to 31 of SEQ ID NO:10; amino acids 1 to 31 of SEQ ID NO:12; amino acids 1 to 23 of SEQ ID NO:14; amino acids 1 to 23 of SEQ ID NO:16; amino acids 1 to 23 of SEQ ID NO:18; amino acids 1 to 31 of SEQ ID NO:20; amino acids 1 to 31 of SEQ ID NO:22; amino acids 1 to 31 of SEQ ID NO:24; amino acids 1 to 27 of SEQ ID NO:26; amino acids 1 to 27 of SEQ ID NO:28; amino acids 1 to 27 of SEQ ID NO:30; amino acids 1 to 31 of SEQ ID NO:32; amino acids 1 to 37 of SEQ ID NO:36; amino acids 1 to 10 of SEQ ID NO:38; amino acids 1 to 33 of SEQ ID NO:40; amino acids 1 to 29 of SEQ ID NO:42; amino acids 1 to 45 of SEQ ID NO:44; amino acids 1 to 27 of SEQ ID NO:46; amino acids 1 to 29 of SEQ ID NO:48; amino acids 1 to 16 of SEQ ID NO:50; amino acids 1 to 7 of SEQ ID NO:54, amino acids 1 to 27 of SEQ ID NO:56; amino acids 1 to 27 of SEQ ID NO:57; amino acids 1 to 27 of SEQ ID NO:58; amino acids 1 to 27 of SEQ ID NO:60; amino acids 1 to 29 of SEQ ID NO:62; amino acids 1 to 33 of SEQ ID NO:70; amino acids 1 to 45 of SEQ ID NO:72; amino acids 1 to 17 of SEQ ID NO:76; amino acids 1 to 17 of SEQ ID NO:78; amino acids 1 to 18 of SEQ ID NO:80; amino acids 1 to 13 of SEQ ID NO:82; amino acids 1 to 13 of SEQ ID NO:84; amino acids 1 to 22 of SEQ ID NO:86; amino acids 1 to 33 of SEQ ID NO:88; amino acids 1 to 18 of SEQ ID NO:96; amino acids 1 to 19 of SEQ ID NO:98; amino acids 1 to 51 of SEQ ID NO:100; amino acids 1 to 54 of SEQ ID NO:110; amino acids 1 to 55 of SEQ ID NO:111; amino acids 1 to 47 of SEQ ID NO:114; amino acids 1 to 25 of SEQ ID NO:116; amino acids 1 to 26 of SEQ ID NO:118; amino acids 1 to 17 of SEQ ID NO:119; amino acids 1 to 17 of SEQ ID NO:121; amino acids 1 to 32 of SEQ ID NO:123, the central conserved domain is selected from the group consisting of amino acids 32 to 129 of SEQ ID NO:2; amino acids 24 to 121 of SEQ ID NO:4; amino acids 28 to 132 of SEQ ID NO:6; amino acids 32 to 129 of SEQ ID NO:8; amino acids 32 to 129 of SEQ ID NO:10; amino acids 32 to 129 of SEQ ID NO:12; amino acids 24 to 121 of SEQ ID NO:14; amino acids 24 to 121 of SEQ ID NO:16; amino acids 24 to 121 of SEQ ID NO:18; amino acids 32 to 136 of SEQ ID NO:20; amino acids 32 to 129 of SEQ ID NO:22; amino acids 32 to 136 of SEQ ID NO:24; amino acids 28 to 132 of SEQ ID NO:26; amino acids 28 to 125 of SEQ ID NO:28; amino acids 28 to 125 of SEQ ID NO:30; amino acids 32 to 129 of SEQ ID NO:32; amino acids 38 to 135 of SEQ ID NO:36; amino acids 11 to 105 of SEQ ID NO:38; amino acids 34 to 138 of SEQ ID NO:40; amino acids 30 to 127 of SEQ ID NO:42; amino acids 46 to 143 of SEQ ID NO:44; amino acids 28 to 125 of SEQ ID NO:46; amino acids 30 to 127 of SEQ ID NO:48; amino acids 17 to 113 of SEQ ID NO:50; amino acids 8 to 102 of SEQ ID NO:54; amino acids 28 to 125 of SEQ ID NO:56; amino acids 28 to 125 of SEQ ID NO:57; amino acids 28 to 125 of SEQ ID NO:58; amino acids 28 to 125 of SEQ ID NO:60; amino acids 30 to 126 of SEQ ID NO:62; amino acids 30 to 127 of SEQ ID NO:64; amino acids 8 to 105 of SEQ ID NO:68; amino acids 34 to 137 of SEQ ID NO:70; amino acids 46 to 143 of SEQ ID NO:72; amino acids 16 to 112 of SEQ ID NO:74; amino acids 18 to 115 of SEQ ID NO:76; amino acids 18 to 115 of SEQ ID NO:78; amino acids 19 to 116 of SEQ ID NO:80; amino acids 14 to 102 of SEQ ID NO:82; amino acids 14 to 104 of SEQ ID NO:84; amino acids 23 to 117 of SEQ ID NO:86; amino acids 34 to 131 of SEQ ID NO:88; amino acids 32 to 129 of SEQ ID NO:92; amino acids 25 to 122 of SEQ ID NO:94; amino acids 19 to 116 of SEQ ID NO:96; amino acids 20 to 117 of SEQ ID NO:98; amino acids 52 to 149 of SEQ ID NO:100; amino acids 12 to 101 of SEQ ID NO:104; amino acids 8 to 105 of SEQ ID NO:106; amino acids 8 to 105 of SEQ ID NO:108; amino acids 3 to 105 of SEQ ID NO:109; amino acids 55 to 152 of SEQ ID NO:110; amino acids 56 to 153 of SEQ ID NO:111; amino acids 1 to 97 of SEQ ID NO:113; amino acids 48 to 145 of SEQ ID NO:114; amino acids 26 to 123 of SEQ ID NO:116; amino acids 27 to 124 of SEQ ID NO:118; amino acids 18 to 115 of SEQ ID NO:119; amino acids 18 to 115 of SEQ ID NO:121; amino acids 33 to 130 of SEQ ID NO:123 and the C-terminal domain is selected from the group consisting of amino acids 130 to 218 of SEQ ID NO:2; amino acids 122 to 190 of SEQ ID NO:4; amino acids 133 to 185 of SEQ ID NO:6; amino acids 130 to 218 of SEQ ID NO:8; amino acids 130 to 198 of SEQ ID NO:10; amino acids 130 to 198 of SEQ ID NO:12; amino acids 122 to 210 of SEQ ID NO:14; amino acids 122 to 190 of SEQ ID NO:16; amino acids 122 to 210 of SEQ ID NO:18; amino acids 137 to 225 of SEQ ID NO:20; amino acids 130 to 182 of SEQ ID NO:22; amino acids 137 to 189 of SEQ ID NO:24; amino acids 133 to 221 of SEQ ID NO:26; amino acids 126 to 178 of SEQ ID NO:28; amino acids 126 to 214 of SEQ ID NO:30; amino acids 130 to 218 of SEQ ID NO:32; amino acids 89 to 150 of SEQ ID NO:34; amino acids 136 to 189 of SEQ ID NO:36; amino acids 106 to 301 of SEQ ID NO:38; amino acids 139 to 175 of SEQ ID NO:40; amino acids 128 to 264 of SEQ ID NO:42; amino acids 144 to 261 of SEQ ID NO:44; amino acids 126 to 178 of SEQ ID NO:46; amino acids 128 to 180 of SEQ ID NO:48; amino acids 114 to 164 of SEQ ID NO:50; amino acids 82 to 165 of SEQ ID NO:52; amino acids 103 to 294 of SEQ ID NO:54; amino acids 126 to 164 of SEQ ID NO:56; amino acids 126 to 178 of SEQ ID NO:57; amino acids 126 to 174 of SEQ ID NO:58; amino acids 126 to 149 of SEQ ID NO:60; amino acids 127 to 154 of SEQ ID NO:62; amino acids 128 to 189 of SEQ ID NO:64; amino acids 85 to 280 of SEQ ID NO:66; amino acids 106 to 297 of SEQ ID NO:68; amino acids 138 to 174 of SEQ ID NO:70; amino acids 144 to 262 of SEQ ID NO:72; amino acids 113 to 187 of SEQ ID NO:74; amino acids 116 to 212 of SEQ ID NO:76; amino acids 116 to 212 of SEQ ID NO:78; amino acids 117 to 118 of SEQ ID NO:80; amino acids 103 to 111 of SEQ ID NO:82; amino acids 105 to 111 of SEQ ID NO:84; amino acids 118 to 197 of SEQ ID NO:86; amino acids 132 to 278 of SEQ ID NO:88; amino acids 130 to 165 of SEQ ID NO:92; amino acids 123 to 178 of SEQ ID NO:94; amino acids 117 to 118 of SEQ ID NO:96; amino acids 118 to 144 of SEQ ID NO:98; amino acids 150 to 259 of SEQ ID NO:100; amino acids 82 to 118 of SEQ ID NO:102; amino acids 102 to 379 of SEQ ID NO:104; amino acids 106 to 163 of SEQ ID NO:106; amino acids 106 to 159 of SEQ ID NO:108; amino acids 106 to 275 of SEQ ID NO:109; amino acids 153 to 234 of SEQ ID NO:110; amino acids 154 to 238 of SEQ ID NO:111; amino acids 98 to 139 of SEQ ID NO:113; amino acids 146 to 160 of SEQ ID NO:114; amino acids 124 to 228 of SEQ ID NO:116; amino acids 125 to 173 of SEQ ID NO:118; amino acids 116 to 141 of SEQ ID NO:119; amino acids 116 to 161 of SEQ ID NO:121; amino acids 131 to 215 of SEQ ID NO:123.

The invention further provides a seed which is true breeding for the expression cassettes (also referred to herein as “transgenes”) described herein, wherein transgenic plants grown from said seed demonstrate increased yield as compared to a wild type variety of the plant. The invention also provides a product produced by or from the transgenic plants expressing the polynucleotide, their plant parts, or their seeds. The product can be obtained using various methods well known in the art. As used herein, the word “product” includes, but not limited to, a foodstuff, feedstuff, a food supplement, feed supplement, fiber, cosmetic or pharmaceutical. Foodstuffs are regarded as compositions used for nutrition or for supplementing nutrition. Animal feedstuffs and animal feed supplements, in particular, are regarded as foodstuffs. The invention further provides an agricultural product produced by any of the transgenic plants, plant parts, and plant seeds. Agricultural products include, but are not limited to, plant extracts, proteins, amino acids, carbohydrates, fats, oils, polymers, vitamins, and the like.

The invention also provides an isolated polynucleotide which has a sequence selected from the group consisting of SEQ ID NO:7; SEQ ID NO:9; SEQ ID NO:11; SEQ ID NO:13; SEQ ID NO:15; SEQ ID NO:17; SEQ ID NO:19; SEQ ID NO:21; SEQ ID NO:23; SEQ ID NO:25; SEQ ID NO:27; SEQ ID NO:29; SEQ ID NO:35; SEQ ID NO:37; SEQ ID NO:39; SEQ ID NO:41; SEQ ID NO:43; SEQ ID NO:45; SEQ ID NO:47; SEQ ID NO:49; SEQ ID NO:51; SEQ ID NO:53. Also encompassed by the isolated polynucleotide of the invention is an isolated polynucleotide encoding a polypeptide having an amino acid sequence selected from the group consisting of SEQ ID NO:8; SEQ ID NO:10; SEQ ID NO:12; SEQ ID NO:14; SEQ ID NO:16; SEQ ID NO:18; SEQ ID NO:20; SEQ ID NO:22; SEQ ID NO:24; SEQ ID NO:26; SEQ ID NO:28; SEQ ID NO:30; SEQ ID NO:34; SEQ ID NO:36; SEQ ID NO:38; SEQ ID NO:40; SEQ ID NO:42; SEQ ID NO:44; SEQ ID NO:46; SEQ ID NO:48; SEQ ID NO:50; SEQ ID NO:52; SEQ ID NO:54. A polynucleotide of the invention can be isolated using standard molecular biology techniques and the sequence information provided herein, for example, using an automated DNA synthesizer.

The chimeric NF-YB polynucleotides and polypeptides of the invention may be constructed using homologs of any plant NF-YB transcription factor. “Homologs” are defined herein as two nucleic acids or polypeptides that have similar, or substantially identical, nucleotide or amino acid sequences, respectively. Homologs include allelic variants, analogs, and orthologs, as defined below. As used herein, the term “analogs” refers to two nucleic acids that have the same or similar function, but that have evolved separately in unrelated organisms. As used herein, the term “orthologs” refers to two nucleic acids from different species, but that have evolved from a common ancestral gene by speciation. The term homolog further encompasses nucleic acid molecules that differ from the NF-YB transcription factor polynucleotides used to produce the chimeric NF-YB transcription factors exemplified in Table 1 due to degeneracy of the genetic code and thus encode the same polypeptide.

To determine the percent sequence identity of two NF-YB transcription factor amino acid sequences (e.g., SEQ ID NO:2; SEQ ID NO:4, or SEQ ID NO:6 of Table 1 and a homolog thereof), the sequences are aligned for optimal comparison purposes (e.g., gaps can be introduced in the sequence of one polypeptide for optimal alignment with the other polypeptide or nucleic acid). The amino acid residues at corresponding amino acid positions are then compared. When a position in one sequence is occupied by the same amino acid residue as the corresponding position in the other sequence then the molecules are identical at that position. The same type of comparison can be made between two nucleic acid sequences.

Preferably, the NF-YB transcription factor amino acid homologs, analogs, and orthologs of the polypeptides of the present invention are at least about 50-60%, preferably at least about 60-70%, and more preferably at least about 70-75%, 75-80%, 80-85%, 85-90%, or 90-95%, and most preferably at least about 96%, 97%, 98%, 99%, or more identical to SEQ ID NO:2, SEQ ID NO:4, or SEQ ID NO:6. In another preferred embodiment, an isolated nucleic acid homolog of the invention comprises a nucleotide sequence which is at least about 40-60%, preferably at least about 60-70%, more preferably at least about 70-75%, 75-80%, 80-85%, 85-90%, or 90-95%, and even more preferably at least about 95%, 96%, 97%, 98%, 99%, or more identical to SEQ ID NO:1, SEQ ID NO:3, or SEQ ID NO:5.

For the purposes of the invention, the percent sequence identity between two nucleic acid or polypeptide sequences is determined using Align 2.0 (Myers and Miller, CABIOS (1989) 4:11-17) with all parameters set to the default settings or the Vector NTI 9.0 (PC) software package (Invitrogen, 1600 Faraday Ave., Carlsbad, Calif. 92008). For percent identity calculated with Vector NTI, a gap opening penalty of 15 and a gap extension penalty of 6.66 are used for determining the percent identity of two nucleic acids. A gap opening penalty of 10 and a gap extension penalty of 0.1 are used for determining the percent identity of two polypeptides. All other parameters are set at the default settings. For purposes of a multiple alignment (Clustal W algorithm), the gap opening penalty is 10, and the gap extension penalty is 0.05 with blosum62 matrix. It is to be understood that for the purposes of determining sequence identity when comparing a DNA sequence to an RNA sequence, a thymidine nucleotide is equivalent to a uracil nucleotide.

Nucleic acid molecules corresponding to homologs, analogs, and orthologs of SEQ ID NO:2, SEQ ID NO:4, or SEQ ID NO:6 can be isolated based on their identity to said polypeptides, using the polynucleotides encoding the respective polypeptides or primers based thereon, as hybridization probes according to standard hybridization techniques under stringent hybridization conditions. As used herein with regard to hybridization for DNA to a DNA blot, the term “stringent conditions” refers to hybridization overnight at 60° C. in 10×Denhart's solution, 6×SSC, 0.5% SDS, and 100 μg/ml denatured salmon sperm DNA. Blots are washed sequentially at 62° C. for 30 minutes each time in 3×SSC/0.1% SDS, followed by 1×SSC/0.1% SDS, and finally 0.1×SSC/0.1% SDS. As also used herein, in a preferred embodiment, the phrase “stringent conditions” refers to hybridization in a 6×SSC solution at 65° C. In another embodiment, “highly stringent conditions” refers to hybridization overnight at 65° C. in 10×Denhart's solution, 6×SSC, 0.5% SDS and 100 μg/ml denatured salmon sperm DNA. Blots are washed sequentially at 65° C. for 30 minutes each time in 3×SSC/0.1% SDS, followed by 1×SSC/0.1% SDS, and finally 0.1×SSC/0.1% SDS. Methods for performing nucleic acid hybridizations are well known in the art.

The isolated polynucleotides employed in the invention may be optimized, that is, genetically engineered to increase its expression in a given plant or animal. To provide plant optimized nucleic acids, the DNA sequence of the gene can be modified to: 1) comprise codons preferred by highly expressed plant genes; 2) comprise an A+T content in nucleotide base composition to that substantially found in plants; 3) form a plant initiation sequence; 4) to eliminate sequences that cause destabilization, inappropriate polyadenylation, degradation and termination of RNA, or that form secondary structure hairpins or RNA splice sites; or 5) elimination of antisense open reading frames. Increased expression of nucleic acids in plants can be achieved by utilizing the distribution frequency of codon usage in plants in general or in a particular plant. Methods for optimizing nucleic acid expression in plants can be found in EPA 0359472; EPA 0385962; PCT Application No. WO 91/16432; U.S. Pat. No. 5,380,831; U.S. Pat. No. 5,436,391; Perlack et al., 1991, Proc. Natl. Acad. Sci. USA 88:3324-3328; and Murray et al., 1989, Nucleic Acids Res. 17:477-498.

In another embodiment, the recombinant expression vector of the invention comprises an isolated polynucleotide having a sequence selected from the group consisting of SEQ ID NO:7; SEQ ID NO:9; SEQ ID NO:11; SEQ ID NO:13; SEQ ID NO:15; SEQ ID NO:17; SEQ ID NO:19; SEQ ID NO:21; SEQ ID NO:23; SEQ ID NO:25; SEQ ID NO:27; SEQ ID NO:29; SEQ ID NO:31; SEQ ID NO:35; SEQ ID NO:37; SEQ ID NO:39; SEQ ID NO:41; SEQ ID NO:43; SEQ ID NO:45; SEQ ID NO:47; SEQ ID NO:49; SEQ ID NO:51; SEQ ID NO:53. In addition, the recombinant expression vector of the invention comprises an isolated polynucleotide encoding a polypeptide having an amino acid sequence selected from the group consisting of SEQ ID NO:8; SEQ ID NO:10; SEQ ID NO:12; SEQ ID NO:14; SEQ ID NO:16; SEQ ID NO:18; SEQ ID NO:20; SEQ ID NO:22; SEQ ID NO:24; SEQ ID NO:26; SEQ ID NO:28; SEQ ID NO:30; SEQ ID NO:34; SEQ ID NO:36; SEQ ID NO:38; SEQ ID NO:40; SEQ ID NO:42; SEQ ID NO:44; SEQ ID NO:46; SEQ ID NO:48; SEQ ID NO:50; SEQ ID NO:52; SEQ ID NO:54.

The recombinant expression vector of the invention includes one or more regulatory sequences, selected on the basis of the host cells to be used for expression, which is in operative association with the isolated polynucleotide to be expressed. As used herein with respect to a recombinant expression vector, “in operative association” or “operatively linked” means that the polynucleotide of interest is linked to the regulatory sequence(s) in a manner which allows for expression of the polynucleotide when the vector is introduced into the host cell (e.g., in a bacterial or plant host cell). The term “regulatory sequence” is intended to include promoters, enhancers, and other expression control elements (e.g., polyadenylation signals).

As set forth above, certain embodiments of the invention employ promoters that are capable of enhancing gene expression in leaves. In some embodiments, the promoter is a leaf-specific promoter. Any leaf-specific promoter may be employed in these embodiments of the invention. Many such promoters are known, for example, the USP promoter from Vicia faba (Baeumlein et al. (1991) Mol. Gen. Genet. 225, 459-67), promoters of light-inducible genes such as ribulose-1.5-bisphosphate carboxylase (rbcS promoters), promoters of genes encoding chlorophyll a/b-binding proteins (Cab), Rubisco activase, B-subunit of chloroplast glyceraldehyde 3-phosphate dehydrogenase from A. thaliana, (Kwon et al. (1994) Plant Physiol. 105, 357-67) and other leaf specific promoters such as those identified in Aleman, I. (2001) Isolation and characterization of leaf-specific promoters from alfalfa (Medicago sativa), Masters thesis, New Mexico State University, Los Cruces, N. Mex.

In other embodiments of the invention, a root or shoot specific promoter is employed. For example, the Super promoter provides high level expression in both root and shoots (Ni et al. (1995) Plant J. 7: 661-676). Other root specific promoters include, without limitation, the TobRB7 promoter (Yamamoto et al. (1991) Plant Cell 3, 371-382), the rolD promoter (Leach et al. (1991) Plant Science 79, 69-76); CaMV 35S Domain A (Benfey et al. (1989) Science 244, 174-181), and the like.

In other embodiments, a constitutive promoter is employed. Constitutive promoters are active under most conditions. Examples of constitutive promoters suitable for use in these embodiments include the parsley ubiquitin promoter described in WO 2003/102198 (SEQ ID NO:65) the CaMV 19S and 35S promoters, the sX CaMV 35S promoter, the Sep1 promoter, the rice actin promoter, the Arabidopsis actin promoter, the maize ubiquitin promoter, pEmu, the figwort mosaic virus 35S promoter, the Smas promoter, the super promoter (U.S. Pat. No. 5,955,646), the GRP1-8 promoter, the cinnamyl alcohol dehydrogenase promoter (U.S. Patent No. 5,683,439), promoters from the T-DNA of Agrobacterium, such as mannopine synthase, nopaline synthase, and octopine synthase, the small subunit of ribulose biphosphate carboxylase (ssuRUBISCO) promoter, and the like.

In a preferred embodiment of the present invention, the polynucleotides listed in Table 1 are expressed in plant cells from higher plants (e.g., the spermatophytes, such as crop plants). A polynucleotide may be “introduced” into a plant cell by any means, including transfection, transformation or transduction, electroporation, particle bombardment, agroinfection, and the like. Suitable methods for transforming or transfecting plant cells are disclosed, for example, using particle bombardment as set forth in U.S. Pat. Nos. 4,945,050; 5,036,006; 5,100,792; 5,302,523; 5,464,765; 5,120,657; 6,084,154; and the like. More preferably, the transgenic corn seed of the invention may be made using Agrobacterium transformation, as described in U.S. Pat. Nos. 5,591,616; 5,731,179; 5,981,840; 5,990,387; 6,162,965; 6,420,630, U.S. patent application publication number 2002/0104132, and the like. Transformation of soybean can be performed using for example any of the techniques described in European Patent No. EP 0424047, U.S. Pat. No. 5,322,783, European Patent No. EP 0397 687, U.S. Pat. No. 5,376,543, or U.S. Pat. No. 5,169,770. A specific example of wheat transformation can be found in PCT Application No. WO 93/07256. Cotton may be transformed using methods disclosed in U.S. Pat. Nos. 5,004,863; 5,159,135; 5,846,797, and the like. Rice may be transformed using methods disclosed in U.S. Pat. Nos. 4,666,844; 5,350,688; 6,153,813; 6,333,449; 6,288,312; 6,365,807; 6,329,571, and the like. Canola may be transformed, for example, using methods such as those disclosed in U.S. Pat. Nos. 5,188,958; 5,463,174; 5,750,871; EP1566443; WO02/00900; and the like. Other plant transformation methods are disclosed, for example, in U.S. Pat. Nos. 5,932,782; 6,153,811; 6,140,553; 5,969,213; 6,020,539, and the like. Any plant transformation method suitable for inserting a transgene into a particular plant may be used in accordance with the invention.

According to the present invention, the introduced polynucleotide may be maintained in the plant cell stably if it is incorporated into a non-chromosomal autonomous replicon or integrated into the plant chromosomes. Alternatively, the introduced polynucleotide may be present on an extra-chromosomal non-replicating vector and may be transiently expressed or transiently active.

The invention is also embodied in a method of producing a transgenic plant comprising at least chimeric NF-YB polynucleotide, wherein expression of the polynucleotide in the plant results in the plant's increased growth and/or yield under normal or water-limited conditions and/or increased tolerance to an environmental stress as compared to a wild type variety of the plant comprising the steps of: (a) introducing into a plant cell an expression cassette described above, (b) regenerating a transgenic plant from the transformed plant cell; and selecting higher-yielding plants from the regenerated plant sells. The plant cell may be, but is not limited to, a protoplast, gamete producing cell, and a cell that regenerates into a whole plant. As used herein, the term “transgenic” refers to any plant, plant cell, callus, plant tissue, or plant part, that contains the expression cassette described above. In accordance with the invention, the expression cassette is stably integrated into a chromosome or stable extra-chromosomal element, so that it is passed on to successive generations.

The effect of the genetic modification on plant growth and/or yield and/or stress tolerance can be assessed by growing the modified plant under normal and/or less than suitable conditions and then analyzing the growth characteristics and/or metabolism of the plant. Such analytical techniques are well known to one skilled in the art, and include measurements of dry weight, wet weight, seed weight, seed number, polypeptide synthesis, carbohydrate synthesis, lipid synthesis, evapotranspiration rates, general plant and/or crop yield, flowering, reproduction, seed setting, root growth, respiration rates, photosynthesis rates, metabolite composition, and the like.

The invention is further illustrated by the following examples, which are not to be construed in any way as imposing limitations upon the scope thereof.

### Example 1

### Structure and Function of NF-YB Transcription Factors

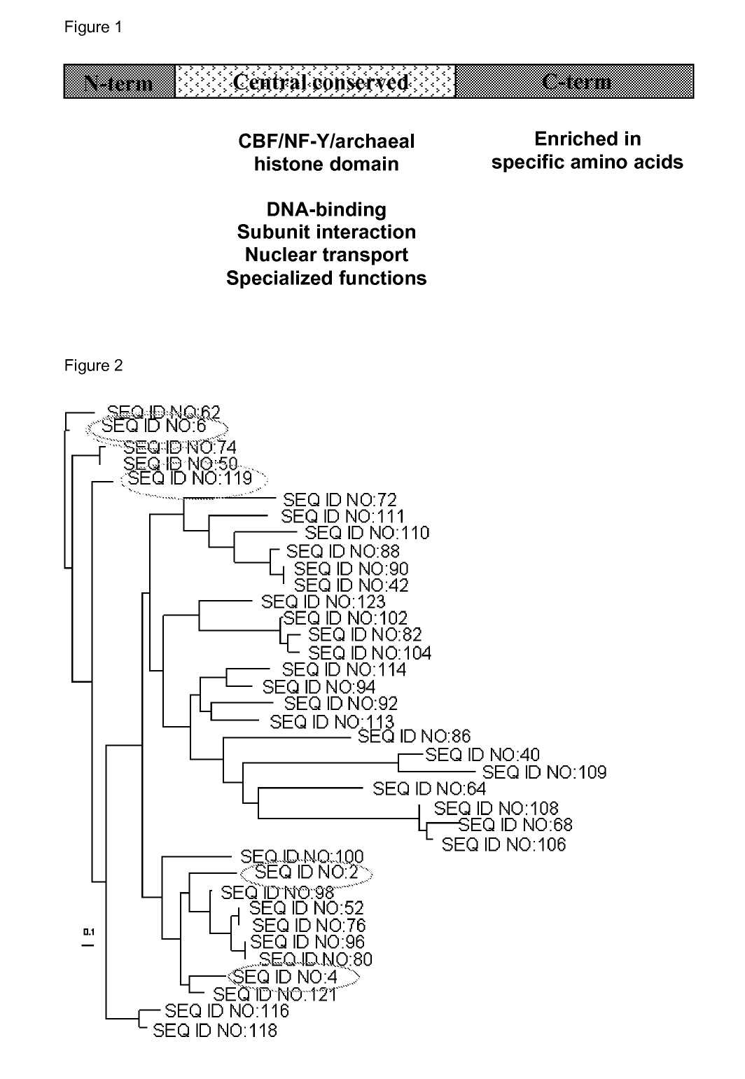

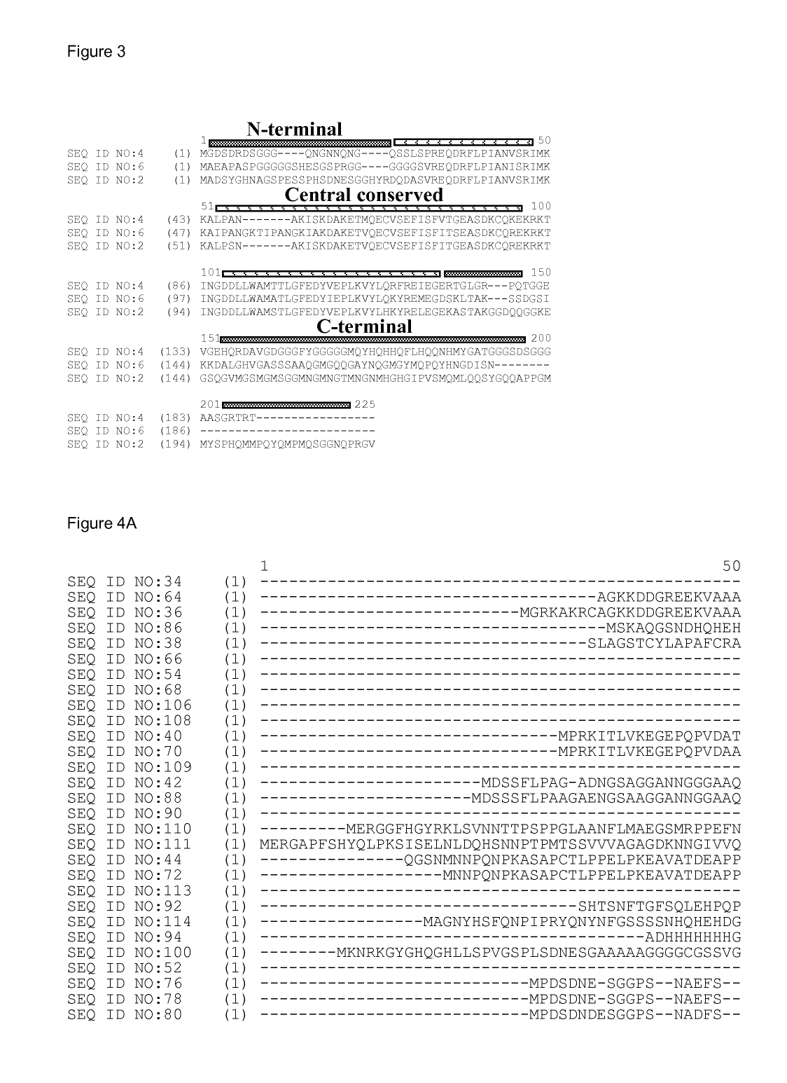

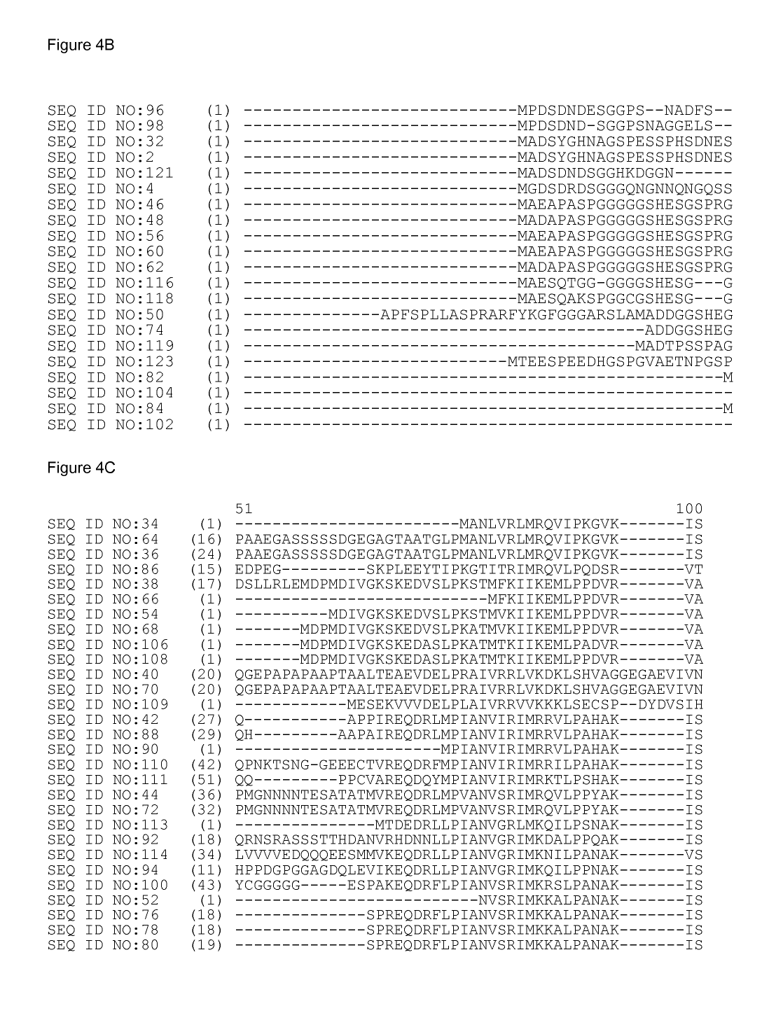

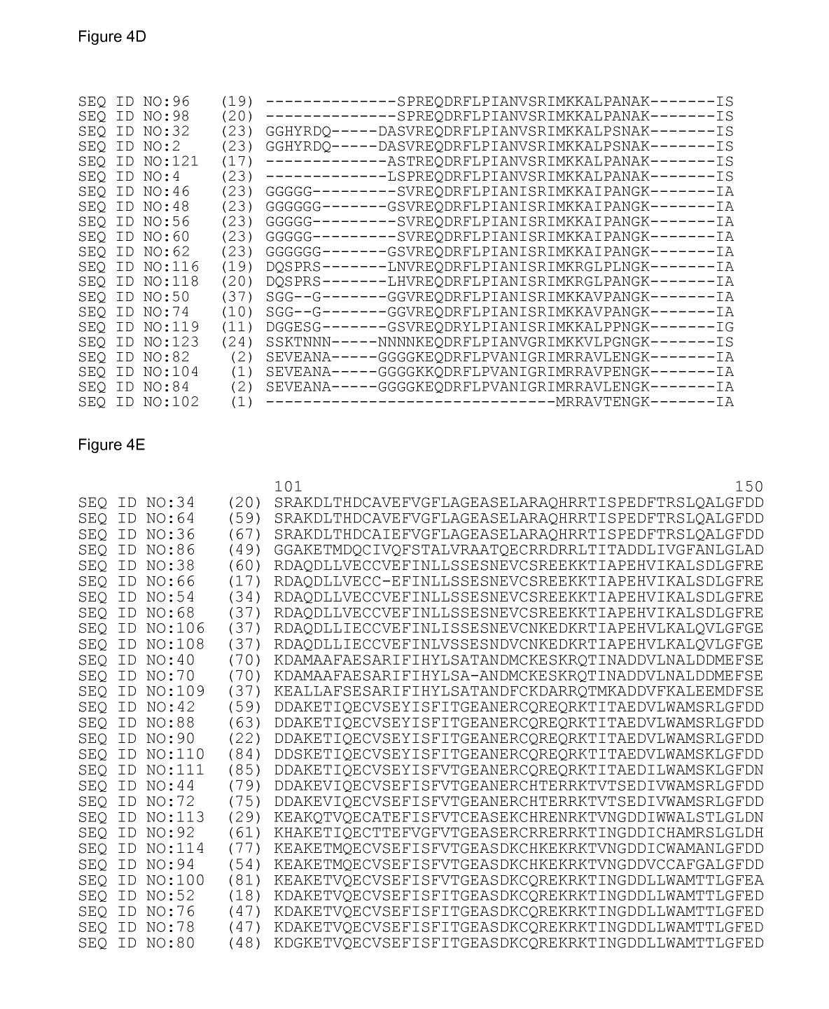

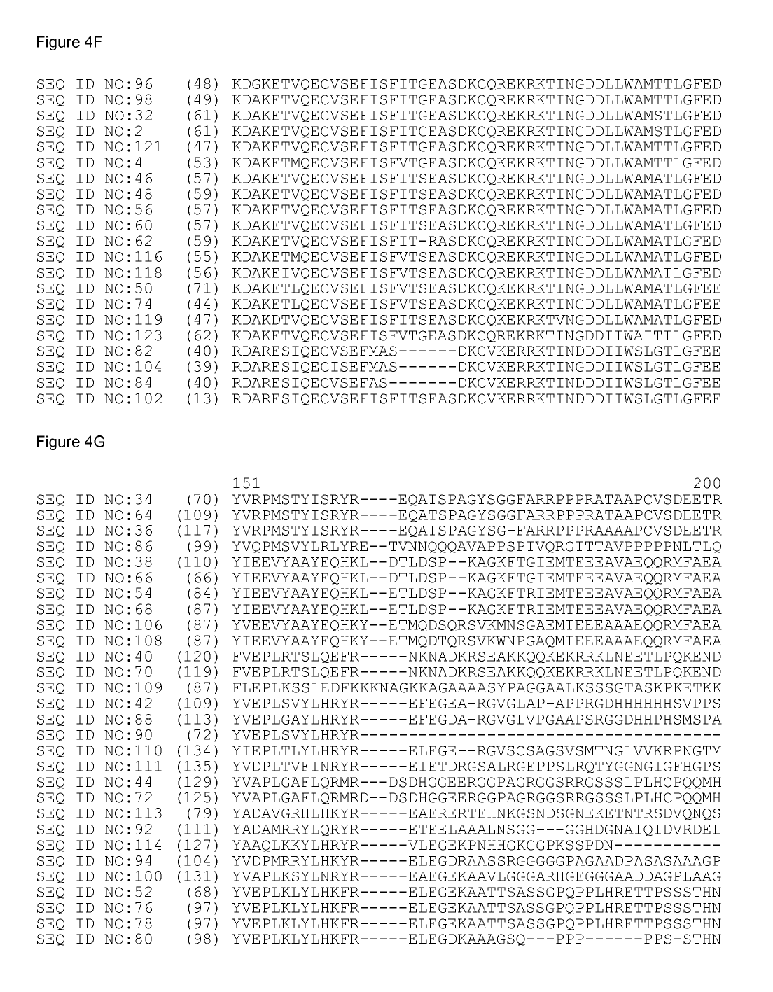

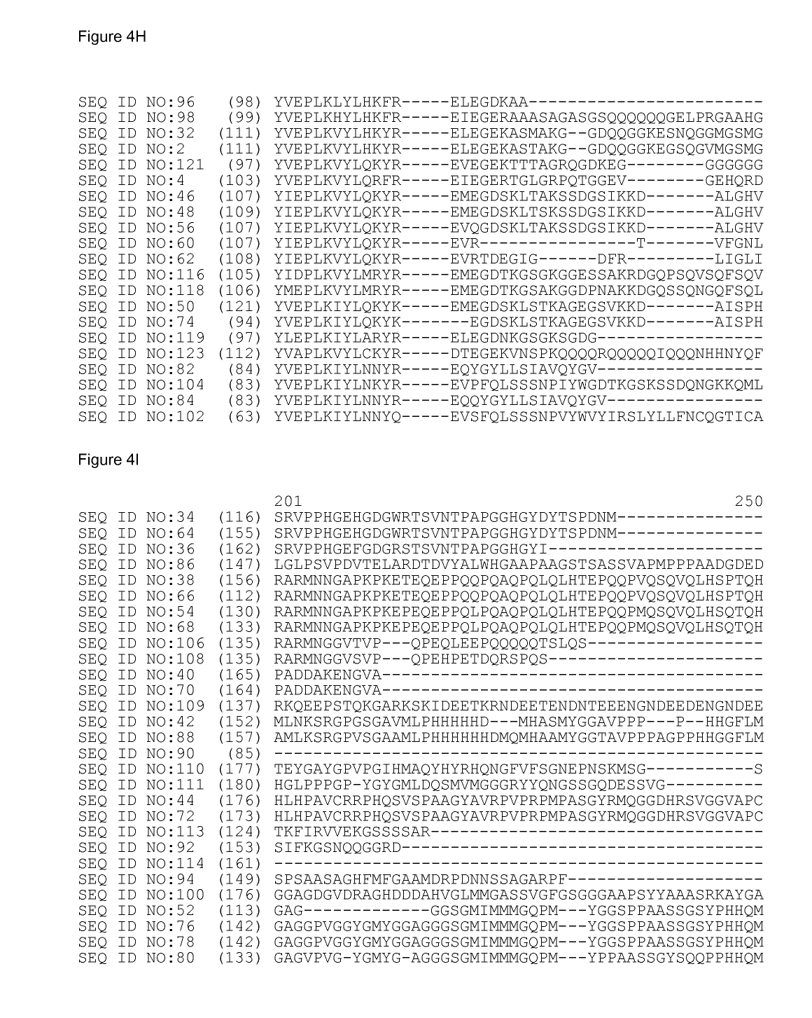

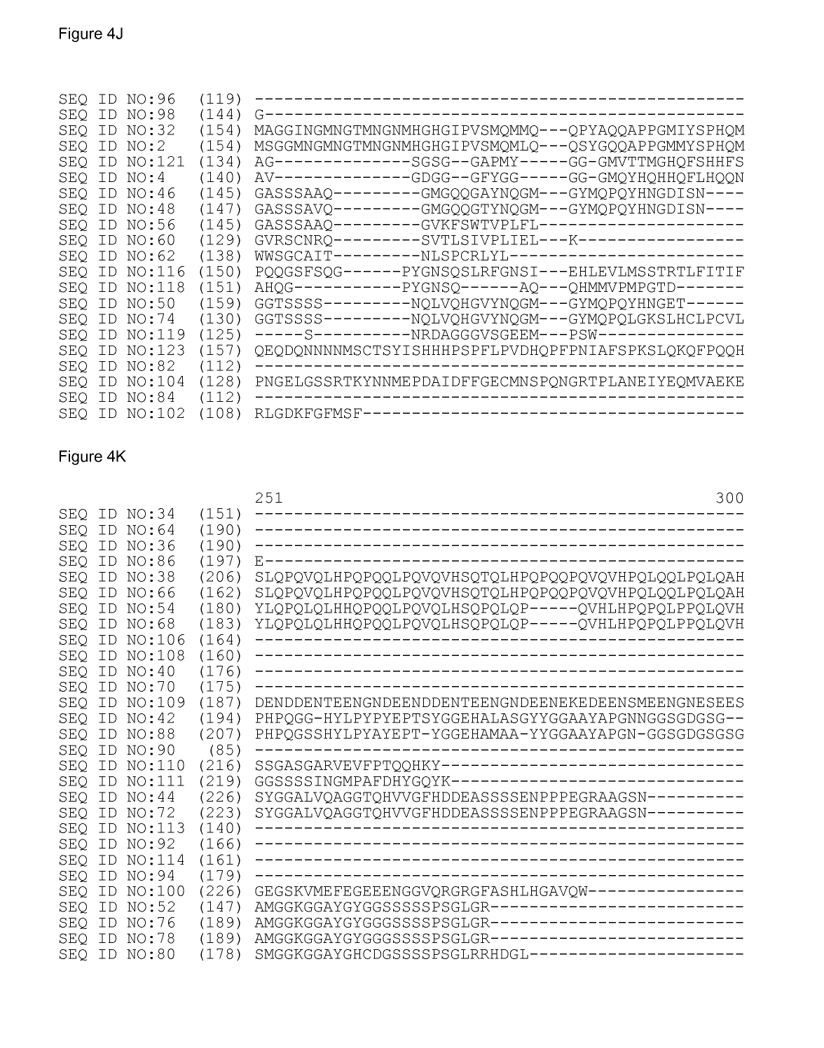

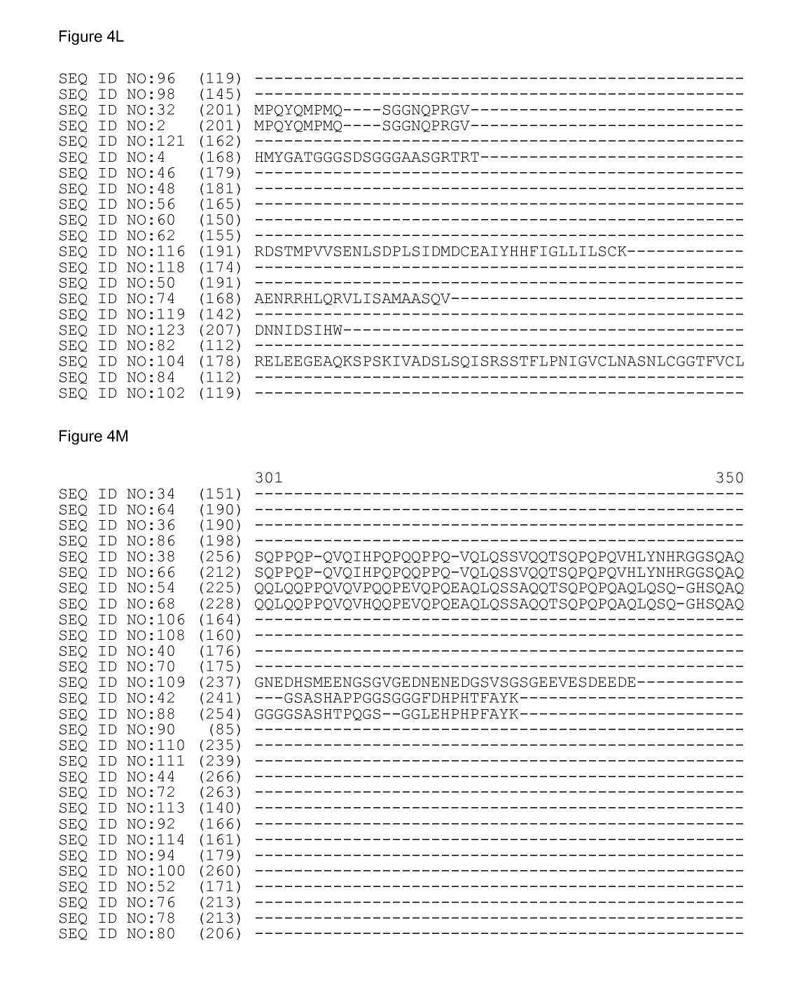

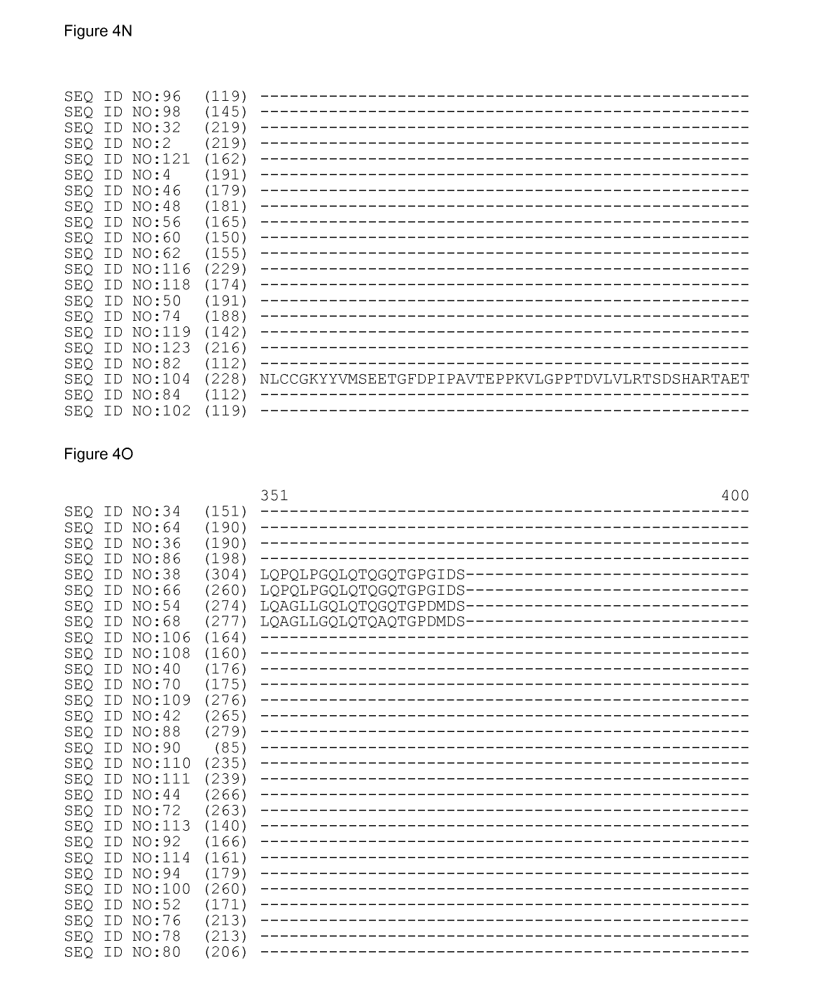

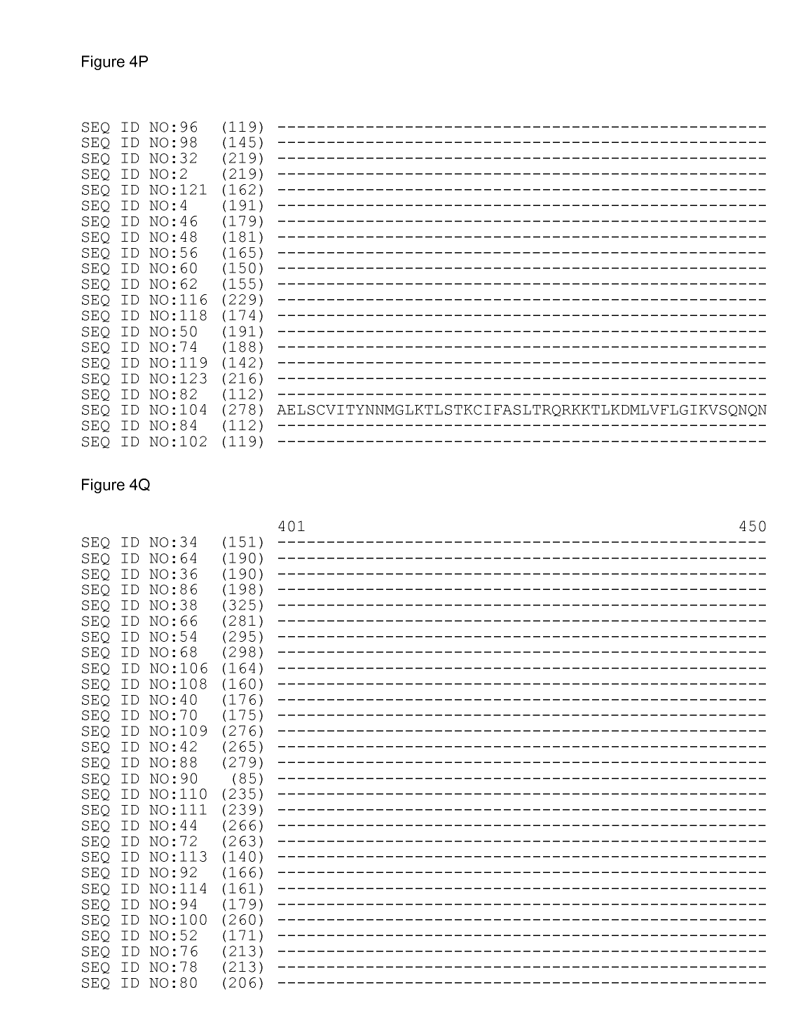

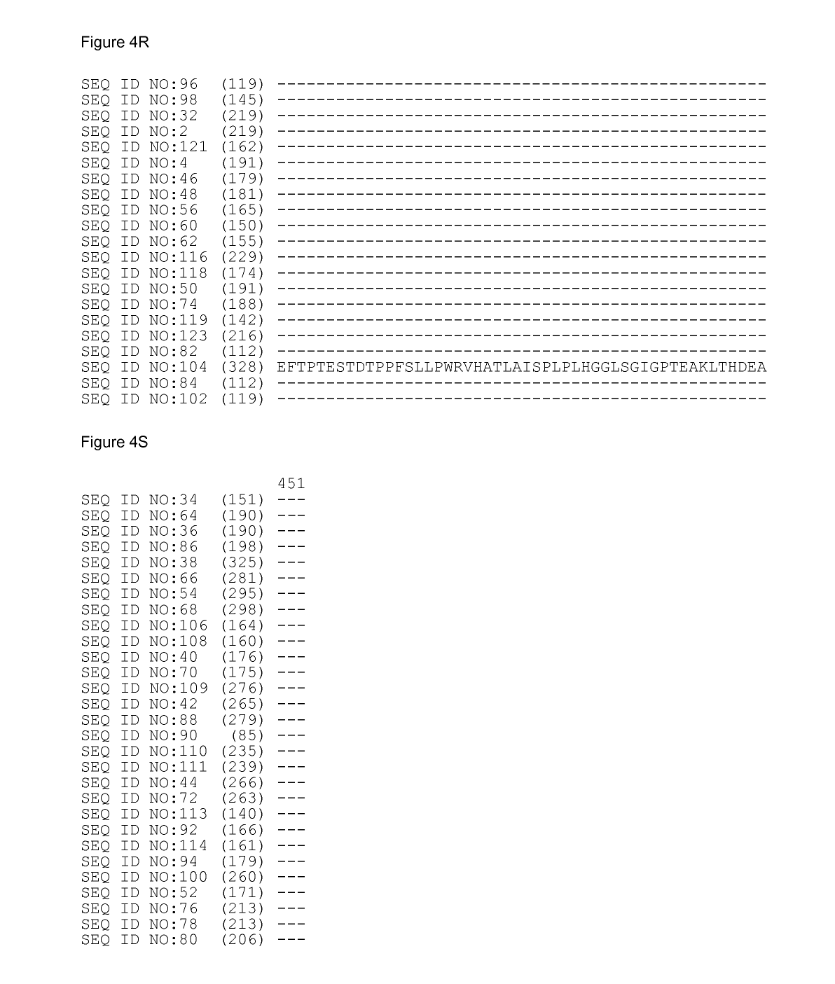

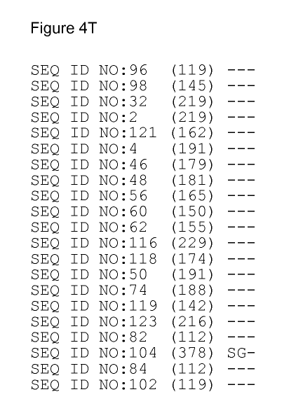

Alignment of the different NF-YB sequences indicate three distinct regions within the NF-YB protein sequences: a divergent N-terminus region, a central conserved domain and divergent C-terminus. The C-terminus region appears to be enriched in specific amino acid residues. FIG. 1 shows the different domains and possible functions. Alignments of the genes are shown in FIGS. 2, 3 and 4.

The P. patens (SEQ ID NO:2), A. thaliana, (SEQ ID NO:4), and maize (SEQ ID NO:6) NF-YB protein sequences were searched using TBLASTN with an e value cutoff of 1e-05. The translated protein sequences were searched against PFAM domain database to check for the conserved domain characteristic of the NF-YB protein. All the genes contained the PFAM PF00808 domain (with the exception of one protein sequence SEQ ID NO:44 that contained homology to PF00125). Also these protein sequences were searched back against public NF-YB sequences including Arabidopsis to confirm that they have identities with these sequences.

Additional maize NF-YB transcription factors listed in Table 1 as SEQ ID NO:34 to SEQ ID NO:54 were searched against the predicted genes from a recent release of the maize genome sequence (Version 2a.50). Table 2 lists proteins that had identities with known NF-YB genes (cutoff of 1e-05 in BLASTP searches) and contained the PFAM PF00808 domain. In addition, SEQ ID NO:44 and SEQ ID NO:72 had homology to PF00125 domain. Table 2 also lists the phylogenetic groups to which each sequence belongs.

TABLE 2

| SEQ ID | Phylogenetic Group |
|---|---|
| 2 | PP |
| 6 | ZM Group 1 |
| 32 | PP |
| 46 | ZM Group 1 |
| 56 | ZM Group 1 |
| 57 | ZM Group 1 |
| 58 | ZM Group 1 |
| 48 | ZM Group 1 |
| 60 | ZM Group 1 |
| 62 | ZM Group 1 |
| 34 | ZM Group 2 |
| 64 | ZM Group 2 |
| 36 | ZM Group 2 |
| 38 | ZM Group 3 |
| 66 | ZM Group 3 |
| 54 | ZM Group 3 |
| 68 | ZM Group 3 |
| 40 | ZM Group 4 |
| 70 | ZM Group 4 |
| 44 | ZM Group 5 |
| 72 | ZM Group 5 |
| 50 | ZM Group 6 |
| 74 | ZM Group 6 |
| 76 | ZM Group 7 |
| 78 | ZM Group 7 |
| 80 | ZM Group 7 |
| 82 | ZM Group 8 |
| 84 | ZM Group 8 |
| 86 | ZM |
| 42 | ZM |
| 88 | ZM |
| 90 | ZM |
| 92 | ZM |
| 94 | ZM |
| 52 | ZM |
| 96 | ZM |
| 98 | ZM |
| 100 | ZM |
| 102 | ZM |
| 104 | ZM |
| 4 | AT |
| 106 | AT |
| 108 | AT |
| 109 | AT |
| 110 | AT |
| 111 | AT |
| 113 | AT |
| 114 | AT |
| 116 | AT |
| 118 | AT |
| 119 | AT |
| 121 | AT |
| 123 | AT |

### Example 2

### Design of Chimeric Genes Based on Functional Domains to Alter Plant Phenotype

Table 3 shows exemplary chimeric genes comprising domains derived from P. patens (SEQ ID NO:2) and A. thaliana (SEQ ID NO:4). Table 4 shows exemplary NFYB chimeric constructs comprising P. patens and maize domains of NF-YB genes. Similar chimeric constructs can be made using NF-YB genes from other plant species. The genes encoding the wild-type NF-YB proteins used in these example chimeric constructs are listed as SEQ ID NO:2, SEQ ID NO:4 and SEQ ID NO:6.

TABLE 3

| | SEQ | | | |
|---|---|---|---|---|
| | ID | | Conserved | |
| Construct | NOs: | N-terminal | central | C-terminal |
| NFYB-C1 | 7/8 | SEQ ID NO: 2 | SEQ ID NO: 4 | SEQ ID NO: 2 |
| | | (CDS 1-93) | (CDS 70-363) | (CDS 388-657) |
| NFYB-C2 | 9/10 | SEQ ID NO: 2 | SEQ ID NO: 2 | SEQ ID NO: 4 |
| | | (CDS 1-93) | (CDS 94-387) | (CDS 364-573) |
| NFYB-C3 | 11/12 | SEQ ID NO: 2 | SEQ ID NO: 4 | SEQ ID NO: 4 |
| | | (CDS 1-93) | (CDS 70-363) | (CDS 364-573) |
| NFYB-C4 | 13/14 | SEQ ID NO: 4 | SEQ ID NO: 4 | SEQ ID NO: 2 |
| | | (CDS 1-69) | (CDS 70-363) | (CDS 388-657) |
| NFYB-C5 | 15/16 | SEQ ID NO: 4 | SEQ ID NO: 2 | SEQ ID NO: 4 |
| | | (CDS 1-69) | (CDS 94-387) | (CDS 364-573) |
| NFYB-C6 | 17/18 | SEQ ID NO: 4 | SEQ ID NO: 2 | SEQ ID NO: 2 |
| | | (CDS 1-69) | (CDS 94-387) | (CDS 388-657) |

TABLE 4

| | SEQ | | | |
|---|---|---|---|---|
| | ID | | Conserved | |
| Construct | NOs: | N-terminal | central | C-terminal |
| NFYB-C7 | 19/20 | SEQ ID NO: 2 | SEQ ID NO: 6 | SEQ ID NO: 2 |
| | | (CDS 1-93) | (CDS 82-396) | (CDS 388-657) |
| NFYB-C8 | 21/22 | SEQ ID NO: 2 | SEQ ID NO: 2 | SEQ ID NO: 6 |
| | | (CDS 1-93) | (CDS 94-387) | (CDS 397-558) |
| NFYB-C9 | 23/24 | SEQ ID NO: 2 | SEQ ID NO: 6 | SEQ ID NO: 6 |
| | | (CDS 1-93) | (CDS 82-396) | (CDS 397-558) |
| NFYB-C10 | 25/26 | SEQ ID NO: 6 | SEQ ID NO: 6 | SEQ ID NO: 2 |
| | | (CDS 1-81) | (CDS 82-396) | (CDS 388-657) |
| NFYB-C11 | 27/28 | SEQ ID NO: 6 | SEQ ID NO: 2 | SEQ ID NO: 6 |
| | | (CDS 1-81) | (CDS 94-387) | (CDS 397-558) |
| NFYB-C12 | 29/30 | SEQ ID NO: 6 | SEQ ID NO: 2 | SEQ ID NO: 2 |
| | | (CDS 1-81) | (CDS 94-387) | (CDS 388-657) |

The genes listed in tables 3 and 4 were ligated into an expression cassette using known methods. The ScBV promoter (SEQ ID NO:124) was used to control expression of the transgenes in Z. mays. A maize inbred was transformed with constructs containing the genes using known methods. Several independent transgenic plants with independent insertion of a single copy of the transgene (events) were grown in the greenhouse and self-pollinated. T1 generation seed was collected from each T0 plant and maintained separately through selection, seed production and phenotype testing. The T1 seed was commonly segregating with a 3:1 Mendelian inheritance of the transgene. Plants were grown in a field nursery from this T1 seed and self-pollinated to create homozygous T2 generation seed. Molecular tests were used to identify transgenic plants using known methods of identification and seed from these T2 generation plants was grown in the greenhouse. Variation is expected to exist among transgenic plants that contain the same transgene, due to different sites of DNA insertion and other factors that impact the level or pattern of gene expression. Therefore multiple events with the same transgene were tested.

Seeds were germinated and plants were grown in the greenhouse under well watered conditions. Images of the transgenic plants were taken at 15 days after planting using a commercial imaging system. Subsequently, plants were grown under a chronic water stress by watering infrequently which allowed the soil to dry between watering treatments. Images of the transgenic plants were taken again at 30 days after planting. The plants were destructively harvested and the shoots were weighed at the end of the experiment.

Image analysis software was used to compare the images of the transgenic and control plants grown in the same experiment. The images were used to determine the relative size of the plants. Images from the top and 2 sides were used to calculate volume. Other measurements including the color of the plants, height, width and area were recorded.

Tables 5 to 7 show the comparison of the size of the maize plants, reported as volume, height and width that was calculated from the images, under well watered and chronic water stress conditions. Percent difference indicates the measurement of the transgenic relative to the control plants as a percentage of the control non-transgenic plants; p-value is the statistical significance of the difference between transgenic and control plants based on a T-test comparison where NS indicates not significant at the 5% level of probability.

Table 5 shows the size (plant volume) of transgenic plants expressing the NF-YB chimeric transgenes under control of the ScBV promoter under well watered conditions at 15 days after planting (before stress) and at 30 days after planting (after stress). Before the water stress, the transgenic plants were smaller than the corresponding non-transgenic control plants for the majority of independent transgenic events. The constructs had different effects on the growth of the plant before and during the stress treatment. The control plants were non-transgenic plants. Variation was also observed among events with the same construct, indicating differences in the site of T-DNA integration or expression of the transgene.

TABLE 5

| | | Before Stress | | After Stress | |
|---|---|---|---|---|---|
| Plant Volume | | Percent | | Percent | |
| Construct ID | Event ID | Difference | P-value | Difference | P-value |
| NFYB-C1 | 104234431 | −9.5 | 0.05 | −1.2 | 0.70 |
| NFYB-C1 | 104234481 | −6.5 | 0.16 | 0.3 | 0.93 |
| NFYB-C1 | 104234531 | −9.1 | 0.05 | −4.1 | 0.17 |
| NFYB-C1 | 104234561 | −16.2 | 0.00 | −8.0 | 0.01 |
| NFYB-C3 | 104071671 | 1.7 | 0.68 | −3.8 | 0.26 |
| NFYB-C3 | 104071681 | −13.6 | 0.00 | −0.1 | 0.97 |
| NFYB-C3 | 104071761 | 8.3 | 0.05 | 0.4 | 0.90 |
| NFYB-C3 | 104071821 | −3.0 | 0.52 | −0.7 | 0.83 |
| NFYB-C3 | 104071971 | 9.9 | 0.02 | 0.5 | 0.87 |
| NFYB-C3 | 104092431 | −43.9 | 0.00 | −4.7 | 0.14 |
| NFYB-C6 | 104369081 | 2.0 | 0.65 | 0.4 | 0.91 |
| NFYB-C6 | 104369121 | −56.7 | 0.00 | −15.1 | 0.00 |
| NFYB-C6 | 104369141 | −55.4 | 0.00 | −14.3 | 0.00 |
| NFYB-C6 | 104381411 | −59.7 | 0.00 | −13.7 | 0.00 |

Table 6 shows the height of transgenic plants expressing the NF-YB chimeric transgenes under control of the ScBV promoter under well watered conditions at 15 days after planting (before stress) and at 30 days after planting (after stress). The constructs had different effects on the growth of the plant before and during the stress treatment. The control plants were non-transgenic plants. Variation was also observed among events with the same construct, indicating differences in the site of T-DNA integration or expression of the transgene.

TABLE 6

| | | Before Stress | | After Stress | |
|---|---|---|---|---|---|
| Plant Height | | Percent | | Percent | |
| Construct ID | Event ID | Difference | P-value | Difference | P-value |
| NFYB-C1 | 104234431 | −7.5 | 0.01 | −1.2 | 0.56 |
| NFYB-C1 | 104234481 | 1.2 | 0.69 | −4.8 | 0.02 |
| NFYB-C1 | 104234531 | −4.4 | 0.15 | −5.2 | 0.01 |
| NFYB-C1 | 104234561 | −4.8 | 0.11 | −6.8 | 0.00 |
| NFYB-C3 | 104071671 | 5.9 | 0.01 | −3.5 | 0.08 |
| NFYB-C3 | 104071681 | −2.4 | 0.26 | 0.8 | 0.70 |
| NFYB-C3 | 104071761 | 4.5 | 0.04 | −1.8 | 0.37 |
| NFYB-C3 | 104071821 | 1.1 | 0.66 | −3.6 | 0.10 |
| NFYB-C3 | 104071971 | 9.3 | 0.00 | −3.5 | 0.09 |
| NFYB-C3 | 104092431 | −12.2 | 0.00 | −0.6 | 0.77 |
| NFYB-C6 | 104369081 | 2.4 | 0.27 | −2.6 | 0.23 |
| NFYB-C6 | 104369121 | −27.7 | 0.00 | −6.3 | 0.00 |
| NFYB-C6 | 104369141 | −27.9 | 0.00 | −7.4 | 0.00 |
| NFYB-C6 | 104381411 | −30.0 | 0.00 | −5.8 | 0.01 |

Table 7 shows the width of transgenic plants expressing the NF-YB chimeric transgenes under control of the ScBV promoter under well watered conditions at 15 days after planting (before stress) and at 30 days after planting (after stress). The constructs had different effects on the growth of the plant before and during the stress treatment. The control plants were non-transgenic plants. Variation was also observed among events with the same construct, indicating differences in the site of T-DNA integration or expression of the transgene.

TABLE 7

| | | Before Stress | | After Stress | |
|---|---|---|---|---|---|
| Plant Width | | Percent | | Percent | |
| Construct ID | Event ID | Difference | P-value | Difference | P-value |
| NFYB-C1 | 104234431 | −5.9 | 0.01 | −2.9 | 0.30 |
| NFYB-C1 | 104234481 | −5.6 | 0.01 | −9.4 | 0.00 |
| NFYB-C1 | 104234531 | −0.7 | 0.70 | 1.7 | 0.52 |
| NFYB-C1 | 104234561 | −13.9 | 0.00 | −5.2 | 0.04 |
| NFYB-C3 | 104071671 | 4.8 | 0.03 | −4.3 | 0.12 |
| NFYB-C3 | 104071681 | −5.7 | 0.01 | −6.4 | 0.03 |
| NFYB-C3 | 104071761 | 2.9 | 0.19 | 1.1 | 0.70 |
| NFYB-C3 | 104071821 | −6.8 | 0.01 | −3.5 | 0.24 |
| NFYB-C3 | 104071971 | 3.5 | 0.12 | −4.9 | 0.08 |
| NFYB-C3 | 104092431 | −25.8 | 0.00 | −1.6 | 0.55 |
| NFYB-C6 | 104369081 | −1.2 | 0.59 | −1.6 | 0.58 |
| NFYB-C6 | 104369121 | −37.8 | 0.00 | −18.2 | 0.00 |
| NFYB-C6 | 104369141 | −28.8 | 0.00 | −16.2 | 0.00 |
| NFYB-C6 | 104381411 | −38.3 | 0.00 | −13.8 | 0.00 |

1. A transgenic plant transformed with an expression cassette comprising, in operative association,
a) an isolated polynucleotide encoding a promoter; and
b) an isolated polynucleotide encoding a full-length polypeptide which is a chimeric NF-YB transcription factor;
wherein the transgenic plant demonstrates increased yield as compared to a wild type plant of the same variety which does not comprise the expression cassette.

2. The transgenic plant of claim 1, wherein the NF-YB polypeptide comprises, in order, i) an N-terminal domain derived from a Physcomitrella patens NF-YB transcription factor, a dicotyledonous plant NF-YB transcription factor, or a monocotyledonous plant NF-YB transcription factor;
ii) a central conserved domain derived from a Physcomitrella patens NF-YB transcription factor, a dicotyledonous plant NF-YB transcription factor, or a monocotyledonous plant NF-YB transcription factor; and
iii) a C-terminal domain derived from a Physcomitrella patens NF-YB transcription factor, a dicotyledonous plant NF-YB transcription factor or a monocotyledonous plant NF-YB transcription factor.

3. The transgenic plant of claim 2, wherein the dicotyledonous plant NF-YB transcription factor is derived from Arabidopsis thaliana.

4. The transgenic plant of claim 2, wherein the monocotyledonous plant NF-YB transcription factor is derived from Zea mays.

5. The transgenic plant of claim 1, further defined as a species selected from the group consisting of maize, wheat, rye, oat, triticale, rice, barley, soybean, peanut, cotton, rapeseed, canola, manihot, pepper, sunflower, tagetes, potato, tobacco, eggplant, tomato, Vicia species, pea, alfalfa, coffee, cacao, tea, Salix species, oil palm, coconut, perennial grasses, and a forage crop plant.

6. A seed which is true breeding for a transgene comprising, in operative association,
a) an isolated polynucleotide encoding a promoter; and
b) an isolated polynucleotide encoding a full-length chimeric NF-YB transcription factor polypeptide;
wherein a transgenic plant grown from said seed demonstrates increased yield as compared to a wild type plant of the same variety which does not comprise the expression cassette.

7. The seed of claim 6, wherein the NF-YB polypeptide comprises, in order,
i) an N-terminal domain derived from a Physcomitrella patens NF-YB transcription factor, a dicotyledonous plant NF-YB transcription factor, or a monocotyledonous plant NF-YB transcription factor;
ii) a central conserved domain derived from a Physcomitrella patens NF-YB transcription factor, a dicotyledonous plant NF-YB transcription factor, or a monocotyledonous plant NF-YB transcription factor; and
iii) a C-terminal domain derived from a Physcomitrella patens NF-YB transcription factor, a dicotyledonous plant NF-YB transcription factor or a monocotyledonous plant NF-YB transcription factor.

8. The seed of claim 7, wherein the dicotyledonous plant NF-YB transcription factor is derived from Arabidopsis thaliana.

9. The seed of claim 8, wherein the monocotyledonous plant NF-YB transcription factor is derived from Zea mays.

10. The seed of claim 7, further defined as a species selected from the group consisting of maize, wheat, rye, oat, triticale, rice, barley, soybean, peanut, cotton, rapeseed, canola, manihot, pepper, sunflower, tagetes, potato, tobacco, eggplant, tomato, Vicia species, pea, alfalfa, coffee, cacao, tea, Salix species, oil palm, coconut, perennial grasses, and a forage crop plant.

11. A method of increasing yield of a plant, the method comprising the steps of:
a) transforming a plant cell with an expression vector comprising, in operative association,
i) an isolated polynucleotide encoding a promoter; and
ii) an isolated polynucleotide encoding a full-length chimeric NF-YB transcription factor polypeptide;
b) regenerating transgenic plants from the transformed plant cell; and
c) selecting drought-tolerant plants from the regenerated transgenic plants.

12. An isolated polynucleotide having a sequence selected from the group consisting of SEQ ID NO:7; SEQ ID NO:9; SEQ ID NO:11; SEQ ID NO:13; SEQ ID NO:15; SEQ ID NO:17; SEQ ID NO:19; SEQ ID NO:21; SEQ ID NO:23; SEQ ID NO:25; SEQ ID NO:27; SEQ ID NO:29; SEQ ID NO:35; SEQ ID NO:37; SEQ ID NO:39; SEQ ID NO:41; SEQ ID NO:43; SEQ ID NO:45; SEQ ID NO:47; SEQ ID NO:49; SEQ ID NO:51; and SEQ ID NO:53.

13. An isolated polynucleotide encoding a polypeptide having an amino acid sequence selected from the group consisting of SEQ ID NO:8; SEQ ID NO:10; SEQ ID NO:12; SEQ ID NO:14; SEQ ID NO:16; SEQ ID NO:18; SEQ ID NO:20; SEQ ID NO:22; SEQ ID NO:24; SEQ ID NO:26; SEQ ID NO:28; SEQ ID NO:30; SEQ ID NO:34; SEQ ID NO:36; SEQ ID NO:38; SEQ ID NO:40; SEQ ID NO:42; SEQ ID NO:44; SEQ ID NO:46; SEQ ID NO:48; SEQ ID NO:50; SEQ ID NO:52; and SEQ ID NO:54.
